(19) United States
(12) Patent Application Publication
(10) Pub. No.: US 2025/0189292 A1
(43) Pub. Date: Jun. 12, 2025

(54) SMART DENDROMETERS FOR TRACKING PLANT GROWTH

(71) Applicant: ePlant, Inc., Los Altos, CA (US)

(72) Inventors: Roger George HINE, Portola Valley, CA (US); Graham Lancaster HINE, Los Gatos, CA (US); Kevin Hugh REA, Menlo Park, CA (US); David B. WALKER, Fremont, CA (US); Kurt A.F. KIESOW, III, San Jose, CA (US); Evan T. DELLOR, La Honda, CA (US)

(73) Assignee: ePlant, Inc., Los Altos, CA (US)

(21) Appl. No.: 18/686,366

(22) PCT Filed: Aug. 31, 2022
(86) PCT No.: PCT/US2022/042152

§ 371 Date: Feb. 23, 2024

Related U.S. Application Data
(60) Provisional application No. 63/239,804, filed on Sep. 1, 2021.
(60) Provisional application No. 63/394,923, filed on Aug. 3, 2022.

(51) Int. Cl. G01B 7/12; A01G 7/00; G01P 15/18

(52) U.S. Cl. CPC G01B 7/12; A01G 7/00; G01P 15/18

(57) ABSTRACT

Described herein are sensors, systems, and methods for measuring plant size, e.g., size of a part of the plant such as a plant stem, trunk, fruit, vine, etc, and/or other plant part characteristics. In some embodiments, the sensor includes two or more components selected from the group consisting of: a dendrometer, an accelerometer, an air temperature sensor, a humidity sensor, and a light sensor. Any of the sensors described herein may relay data to a mobile device or server to inform users of plant health and/or map the connectivity of a wireless network of sensors.

### CROSS-REFERENCE TO RELATED APPLICATIONS

This application claims the priority benefit of U.S. Provisional Application Ser. No. 63/239,804, filed Sep. 1, 2021, and U.S. Provisional Application Ser. No. 63/394,923, filed Aug. 3, 2022, each of which is hereby incorporated by reference in its entirety.

### FIELD

The present disclosure relates generally to monitoring the growth and/or other characteristics of plants and/or plant parts.

### BACKGROUND

Dendrometers are used to measure the size of parts of a plant, usually the stem, trunk or fruit. They have primarily been a research tool but because of the richness of the information that can be gained from these measurements, routine use by farmers is starting to occur.

Two types of dendrometer are common: band dendrometers and point dendrometers. Band dendrometers measure the circumference of a plant stem/trunk—usually a tree—and can be simple tapes with no electronics that are viewed by a person looking at a scale or using calipers or another device to measure the change in tape end locations over time. Other band dendrometers use an electronic instrument to measure band movement automatically and transfer this data to an electronic data logger. Point dendrometers typically anchor in the relatively stationary, relatively dead xylem or woody tissue of the tree and use a precise linear gage such as an linear variable differential transformer (LVDT) to measure the thickness of the living tissue beneath the bark.

These low-tech dendrometers provide scarce data and require significant effort and attention to monitor. As such, there is a need for improved dendrometers, e.g., for measuring plant growth over time, including in real time. These dendrometers allow for both short-term and long-term monitoring of plant growth and are able to interface with other devices (such as mobile devices including smartphones), thus providing rich data on plant growth to a variety of users with an inexpensive and easy-to-manufacture device.

### BRIEF SUMMARY

Provided herein inter alia are “smart” dendrometers that allow farmers, gardeners, landscapers, municipal plant managers, land managers, forest managers or anyone to monitor the growth of a plant over short and long periods. These devices can show the change in plant size that may occur due to sap flow as well as growth over the course of a day, hour, or even a few seconds to minutes. Over longer terms, these devices can provide data as to the health of the plant and if intervention may be needed. These devices, which are low-cost to manufacture, can be installed for long periods of time without maintenance, can be sealed for the life of the device, do not require battery replacement for the life of the device, and can provide a variety of real-time data on size changes (down to micron resolution) as well as temperature, humidity, light, and so forth. Moreover, as described herein, they can be fitted to a variety of plant types and parts.

To achieve these goals and make them possible for widespread use, provided herein are devices that are very low cost and can precisely measure plant part diameters of a wide variety of plants of many sizes. These devices can also transfer that data to a mobile device, server, or other computer system (e.g., wirelessly, directly, or via a network/server) that makes the data available easily and in a way that can be used simply to make decisions or as part of an automatic control system for irrigation or fertilization.

In certain aspects, provided herein are sensors for measuring plant part size and/or other plant part characteristics, comprising: one or more fasteners configured to be positioned in or around a plant part; two or more components selected from the group consisting of: a dendrometer, an accelerometer, an air temperature sensor, a humidity sensor, and a light sensor; a processor; and a power supply.

In some embodiments, the processor comprises a printed circuit board (PCB). In some embodiments, one or both of the two or more components is/are affixed to the PCB. In some embodiments, all of the two or more components are affixed to the PCB. In some embodiments, the PCB comprises an epoxy-fiberglass composite material.

In some embodiments, the power supply comprises a battery. In some embodiments, the battery is a coin cell battery. In some embodiments, the battery is affixed to the PCB. In some embodiments, the power supply comprises a solar panel. In some embodiments, the power supply comprises an integrated solar panel, hybrid capacitor, and lithium battery. In some embodiments, the solar panel is affixed to the PCB.

In some embodiments, the sensor further comprises a housing, e.g., that encloses at least the processor and power supply. In some embodiments, the housing is or comprises plastic, e.g., molded plastic. In some embodiments, the housing is or comprises a polymer resin. In some embodiments, the plastic or polymer resin is glass-filled. In some embodiments, the plastic or polymer resin comprises about 10 to about 40% glass, e.g., about 30% glass. In some embodiments, the processor and magnetometer are enclosed in a sealed, overmolded housing comprising an O-ring. In some embodiments, the overmolded housing comprises a removable lid covering the battery. In some embodiments, the housing is a single piece of overmolded plastic that lacks a seal, junction, or fastener.

In some embodiments, the sensor comprises a dendrometer. In some embodiments, the dendrometer comprises: a plunger having a cap and a shaft, wherein the cap is configured to be positioned against the plant part, and wherein the plunger is configured to move laterally in proportion to a change in plant size when the cap is positioned against the plant part; a magnet attached to or within the shaft, wherein the magnet is configured to move laterally in association with the plunger; and a magnetometer configured to detect position of the magnet. In some embodiments, the magnetometer is configured to detect position of the magnet along multiple axes, a radial axis, or a single plane. In some embodiments, the magnetometer is configured to detect position of the magnet at micron-scale resolution. In some embodiments, the magnetometer is configured to detect position of the magnet along multiple axes, e.g., along a radial axis. In some embodiments, the magnetometer is configured to detect position of the magnet using a ratiometric measurement.

In some embodiments, the sensor is configured to measure change in diameter or radius of the plant part. In some embodiments, the sensor is configured to measure plant part size multiple times per day or at an interval of 15 minutes, 5 minutes, 5 seconds, between 5 seconds and 1 hour, or between 5 seconds and 15 minutes. In some embodiments, the magnet is neodymium magnet. In some embodiments, the processor comprises a PCB, and wherein the magnetometer is affixed to the PCB.

In some embodiments, the sensor comprises an accelerometer. In some embodiments, the accelerometer is a 3-axis accelerometer. In some embodiments, the processor comprises a PCB, and the accelerometer is affixed to the PCB. In some embodiments, the sensor comprises a light sensor. In some embodiments, the processor comprises a PCB, and the light sensor is affixed to the PCB. In some embodiments, the sensor comprises a humidity sensor. In some embodiments, the processor comprises a PCB, and the humidity sensor is affixed to the PCB. In some embodiments, the sensor comprises an air temperature sensor. In some embodiments, the processor comprises a PCB, and the air temperature sensor is affixed to the PCB.

In some embodiments, the sensor comprises a dendrometer and one or more of: an accelerometer, an air temperature sensor, a humidity sensor, and a light sensor. In some embodiments, the sensor comprises a dendrometer, an accelerometer, an air temperature sensor, a humidity sensor, and a light sensor.

In some embodiments, the sensor further comprises a transmitter or transceiver. In some embodiments, the transmitter is a Bluetooth radio or transceiver, e.g., a Bluetooth Low Energy (BLE) radio or transceiver. In some embodiments, the transmitter is a Long Range (LoRa) transceiver. In some embodiments, the transmitter is a Near Field Communication (NFC) transceiver. In some embodiments, the transmitter is affixed to the PCB.

In some embodiments, the one or more fasteners comprises a screw, threaded rod, or nail, and wherein the screw, threaded rod, or nail is configured to be positioned within the plant part and mount the sensor to the plant part. In some embodiments, the one or more fasteners comprises one or more curved arm(s), wherein the curved arm(s) are configured to be positioned around the plant part. In some embodiments, the one or more fasteners comprises two curved arms arranged in a U- or V-shape. In some embodiments, the curved arm(s) are configured to be positioned around the plant part opposite the plunger cap. In some embodiments, the one or more fasteners further comprises an elastic band configured to be wrapped around the sensor and the plant part. In some embodiments, the screw, threaded rod, or nail comprises stainless steel, brass, aluminum, or titanium. In some embodiments, the sensor further comprises a nut configured to be positioned around the screw between the sensor and the plant part. In some embodiments, the sensor further comprises a second nut configured to be positioned around the screw on a face of the sensor distal to the plant part. In some embodiments, the one or more fasteners comprises a screw having a first end and a second end, and the sensor further comprises a compression-limiting element having a first opening and a second opening; and a captive screw; wherein the first end of the screw is configured to be positioned within the plant part and mount the sensor to the plant part; wherein the first opening of the compression-limiting element is configured to receive the second end of the screw; and wherein the second opening of the compression-limiting element is configured to receive the captive screw. In some embodiments, the sensor further comprises a retaining ring configured to be positioned around the captive screw. In some embodiments, the sensor further comprises a first nut configured to be positioned around the threaded rod between the plant part and the sensor and a second nut configured to be positioned around the threaded rod adjacent to the sensor and distal to the plant part. In some embodiments, the sensor further comprises a hollow shuttle positioned around the plunger shaft. In some embodiments, the plunger cap further comprises a gimbal. In some embodiments, the plunger cap is or comprises molded plastic. In some embodiments, the plunger cap is less than about 3 mm in thickness. In some embodiments, the plunger cap is configured to contact the plant part over a surface area of between about 10 mm² and about 100 mm². In some embodiments, the sensor further comprises a spring around or affixed to the plunger. In some embodiments, the sensor further comprises a pull tab attached to the plunger shaft opposite the plunger cap. In some embodiments, the plunger shaft comprises aluminum or stainless steel. In some embodiments, the plunger shaft is a partly or fully hollow cylinder, and the magnet is a cylindrical magnet positioned inside the plunger shaft.

In some embodiments, the plant is a tree or woody plant. In some embodiments, the plant part is a stem, trunk, bole, or branch. In some embodiments, the plant is a crop tree. In some embodiments, the plant is a citrus, olive, nut, cacao, oak, pine, redwood, “strawberry,” or maple tree. In some embodiments, the plant is a vine. In some embodiments, the plant part is a trunk, shoot, branch, cane, fruit, or stem. In some embodiments, the vine is a grape vine.

In certain aspects, provided herein are sensors for measuring plant part size, comprising: a) one or more fasteners configured to be positioned around a plant part, wherein the one or more fasteners comprise(s) a rotatable element, and the rotatable element is configured to rotate in proportion to a change in plant size when positioned around a plant part; b) a magnet, wherein the magnet is configured to rotate in accordance with the rotatable element; c) a rotational sensor configured to detect rotation of the magnet; d) a processor; and e) a power supply.

In some embodiments according to any of the embodiments described herein, the magnet is configured such that a North-South pole axis of the magnet is perpendicular to a rotational axis of the rotatable element. In some embodiments, the rotational sensor is a Hall sensor. In some embodiments, the Hall sensor is positioned such that a Z-axis of the Hall sensor is parallel with a rotational axis of the rotatable element. In some embodiments, the degree of rotation of the rotatable element is linear relative to plant part size by a constant factor. In some embodiments, the constant factor is about 10 degrees of rotation of the rotatable element per about 1 mm of plant part size change. In some embodiments, the constant factor is constant over a dynamic range of plant part size. In some embodiments, the dynamic range of plant part size is from about 4 mm to 24 mm in diameter.

In some embodiments, the one or more fasteners comprise(s) at least a first stationary arm having a base and a rotatable arm having a base, wherein the magnet is positioned within the rotatable arm, and wherein change in size of the plant part causes rotation of the rotatable arm. In some embodiments, the at least first stationary arm and rotatable arm are curved. In some embodiments, the at least first stationary arm and rotatable arm are curved in opposing directions. In some embodiments, the plant part is contacted by three lines of contact, wherein first line is on the first stationary arm, wherein the second line is on the rotatable arm, and wherein the third line is on the sensor opposite the first and/or second line(s). In some embodiments, the sensor further comprises a torsion spring, wherein the torsion spring is connected to the first stationary arm and the rotatable arm. In some embodiments, the base of the rotating arm and the base of the first stationary arm are connected at a hinge comprising the torsion spring. In some embodiments, the position of the base of the first stationary arm is configured to slide relative to the base of the rotational arm, such that sliding the base of the first stationary arm a greater distance from the base of the rotational arm causes an increase in the minimum diameter that can be measured by the sensor and a decrease in the minimum change in size that can be measured by the sensor. In some embodiments, the rotational sensor is positioned within a housing of the sensor. In some embodiments, the one or more fasteners further comprise a second stationary arm. In some embodiments, the rotational sensor is positioned within the second stationary arm.

In some embodiments, the one or more fasteners comprise(s) a clip and a flexible tape with a first end and a second end; wherein the first end is attached to a rotatable drum, wherein the magnet is positioned within the rotatable drum; wherein the second end is configured to be attached with the clip to the sensor; wherein a first section of the flexible tape comprising the first end is configured to be spooled around the rotatable drum; wherein a second section of the flexible tape comprising the second end is configured to be wrapped around the plant part and attached to the sensor with the clip at the second end; and wherein the rotatable drum is configured to rotate in proportion to the change in size of the plant part. In some embodiments, the flexible tape comprises a perforated material, polyethylene terephthalate glycol (PETG), a fluorinated material, a composite material, or any combination thereof. In some embodiments, the composite material comprises Kevlar, fiberglass, or a combination thereof.

In some embodiments, the one or more fasteners comprise(s) a ribbon, a clasp, and a rotatable drum, wherein the magnet is positioned within the rotatable drum; wherein the ribbon is configured to be wrapped around the plant part and fastened to the sensor with the clasp; wherein the rotatable drum is configured to rotate in proportion to the change in change in size of the plant part. In some embodiments, the sensor further comprises a torsion spring; wherein the torsion spring is connected to the rotatable drum; and wherein the torsion spring applies torsion to the rotatable drum or a connection to the sensor thereto.

In some embodiments, the one or more fasteners comprise(s) a belt with a plurality of teeth, a clasp, and a toothed pulley, wherein the magnet is positioned within the toothed pulley; wherein the belt is configured to be wrapped around the plant part and fastened to the sensor with the clasp; wherein the toothed pulley is configured to interlock with one or more of the teeth of the belt and rotate in proportion to the change in size of the plant part. In some embodiments, the belt comprises Kevlar, metal, fiberglass fibers, or a combination thereof. In some embodiments, the teeth are spaced about 2 mm apart. In some embodiments, the rotational sensor is positioned within a housing of the sensor.

In some embodiments according to any of the embodiments described herein, the sensor further comprises a transmitter. In some embodiments, the transmitter is a Bluetooth radio or transceiver, e.g., a Bluetooth Low Energy (BLE) radio or transceiver. In some embodiments, the sensor further comprises a housing. In some embodiments, the housing is or comprises molded plastic. In some embodiments, the rotational sensor, the processor, and/or the power supply are positioned within the housing. In some embodiments, the power supply comprises a battery and/or a solar panel. In some embodiments, the processor comprises a printed circuit board (PCB). In some embodiments, the sensor further comprises a visual identifier. In some embodiments, the visual identifier is a QR code or a bar code. In some embodiments, the sensor further comprises a radio-frequency identification (RFID) tag. In some embodiments, the plant part is a stem, bole, shoot, cane, body, branch, vine, trunk, or fruit of the plant.

In other aspects, provided herein are systems for measuring plant part size and/or other plant part characteristics, comprising: a sensor according to any one of the above embodiments; and a mobile device or server; wherein the sensor is connected to the mobile device or server via wireless communication and configured to transmit data to the mobile device or server. In some embodiments, the sensor is connected to the mobile device or server via Bluetooth low energy (BLE), Long Range (LoRa), or a combination thereof. In some embodiments, the sensor is configured to transmit data to the mobile device or server. In some embodiments, the sensor is configured to transmit data related to the rotational sensor, plant part size, wireless communication signal strength, or a combination thereof to the mobile device or server. In some embodiments, the system comprises a plurality of sensors according to any one of the above embodiments; wherein each sensor of the plurality is connected to the mobile device or server via wireless communication and configured to transmit data to the mobile device or server. In some embodiments, each sensor of the plurality is connected to the mobile device or server via Bluetooth low energy (BLE), Long Range (LoRa), or a combination thereof. In some embodiments, each sensor in the plurality is configured to transmit data related to wireless communication signal strength to the mobile device or server. In some embodiments, the mobile device comprises a GPS sensor. In some embodiments, the GPS sensor is configured to obtain location information using the GPS sensor and associate the location information with a sensor of the plurality. In some embodiments, the mobile device comprises a camera or other image sensor. In some embodiments, the sensor is configured to transmit data related to one or more of: the magnetometer, plant part size, wireless communication signal strength, accelerometer, light sensor, humidity sensor, air temperature sensor, or a combination thereof to the mobile device and/or server. In some embodiments, the system further comprises a server, wherein each sensor of the plurality is connected to the server and configured to transmit data to the mobile device.

In other aspects, provided herein is a method for tracking plant part size and/or other plant part characteristics, comprising: measuring size and/or other plant part characteristics of the plant part using a sensor of the present disclosure, wherein the measurement is based at least in part on data collected by the component(s) of the sensor. In some embodiments, the method comprises, prior to the measurement, mounting the sensor to the plant or plant part, wherein the one or more fasteners is/are positioned in or around the plant part. In some embodiments, the method further comprises measuring size and/or other plant part characteristics of the plant part using a sensor of the present disclosure at a second time after the first time, wherein the measurement of size and/or other plant part characteristics at the second time is based at least in part on data collected by the component(s) of the sensor.

In other aspects, provided herein are methods for tracking plant part size and/or other plant part characteristics, comprising: a) at a first time, measuring plant part size and/or other plant part characteristics at a sensor or system according to any one of the above embodiments; and b) at a second time after the first time, measuring plant part size and/or other plant part characteristics at the sensor or system. In some embodiments, the methods comprise measuring size and/or other plant part characteristics of a plurality of plant parts, e.g., using a system of the present disclosure.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

The present application can be understood by reference to the following description taken in conjunction with the accompanying figures.

FIG. 1A depicts a vertical section view of a clip dendrometer, in accordance with some embodiments.

FIG. 1B depicts top views of a clip dendrometer with three sized stems, in accordance with some embodiments. Dots represent nominal lines of contact with a cylindrical object. Three contacts provide kinematically stable grip at all stem sizes within range. The curvature of arms shown produces a consistent ratio of angular arm movement versus stem diameter change. Ten degrees equals one millimeter over a range of stems from 4 millimeters to 24 millimeters in diameter. A knurled finger tap enables easy opening of clip arms with one hand.

FIG. 1C depicts a clip dendrometer on a plant.

FIG. 2A depicts a horizontal section view of a tape dendrometer, in accordance with some embodiments.

FIG. 2B depicts a vertical section view of a tape dendrometer, in accordance with some embodiments.

FIG. 2C depicts a tape dendrometer on a plant.

FIG. 3A depicts three views of a ribbon dendrometer, in accordance with some embodiments. Flared support arms cradle smaller stems in v-shaped sections and transition to curved portions on larger diameter trunks. One device is stable on a very wide variety of stem diameters.

FIG. 3B depicts a ribbon dendrometer on a potted plant. Extra ribbon allows a dendrometer to be installed on a much larger plant. A small drum diameter results in high measurement sensitivity. A ribbon is pulled snug and then a friction clip holds ribbon in place. Ribbon pulls device toward plant while v-support keeps sensor from rocking.

FIG. 4A depicts a perspective view and two section views of a timing belt dendrometer, in accordance with some embodiments. Upper and lower flared V arms are for stable positioning on stem/trunk. Rotating clip is for easy fastening of timing belt at desired location. Pulley with teeth to engage timing belt. Spring resists rotation and magnet is fixed in lower end of pulley above hall sensor on PCB in sealed housing.

FIG. 4B depicts a timing belt dendrometer with a clip in partly open and open positions. Retention teeth engage belt to secure it.

FIG. 4C depicts a timing belt dendrometer on a tree.

FIG. 5 depicts the measured change in diameter of six tomato plant stems, one rubber plant stem, and one reference cylinder using a mixture of clip-style (ed) and band-style™ dendrometers. One tomato plant was measured using two dendrometers, where one dendrometer was positioned directly above another on the stem (ed3 and ed4).

FIG. 6 depicts the measured change in diameter of a Fuyu Persimmon tree as its water levels fluctuated over approximately three days. The diameter was measured and reported every 30 seconds using a tape-style dendrometer.

FIG. 7A depicts the measured change in diameter of six trees, the air temperature, and the relative humidity during a measurement period.

FIG. 7B depicts the measured change in the magnetometer temperature, the battery level, and the light intensity during a measurement period.

FIG. 7C depicts the measured change in the accelerometer x-axis, y-axis, and z-axis during a measurement period.

FIG. 8A depicts the measured change in diameter of one tree (top panel), the air temperature (middle panel), and the relative humidity (bottom panel) during a measurement period.

FIG. 8B depicts the measured change in the magnetometer temperature (top panel), the battery level (middle panel), and the light intensity (bottom panel) during a measurement period.

FIG. 8C depicts the measured change in the accelerometer x-axis (top panel), y-axis (middle panel), and z-axis (bottom panel) during a measurement period.

FIG. 9A depicts a device measuring the diameter of a tree. The device comprises a plunger, magnetometer (size), accelerometer (lean), antenna, and components that measure humidity, temperature, and light spectrum. The tree comprises bark (cork), growth layer (phloem), and hardwood (xylem).

FIG. 9B depicts a device measuring the diameter of a tree after its diameter has increased. The device comprises a plunger, magnetometer (size), accelerometer (lean), antenna, and components that measure humidity, temperature, and light spectrum. The tree comprises bark (cork), growth layer (phloem), and hardwood (xylem). Arrow indicates lateral movement of the plunger as the tree diameter increases.

FIG. 10 depicts the measured change in diameter of a lime tree over a two-month period. Daily maximum (early am), daily minimum (late day), daily variation, tree water deficit (TWD), and the size of a human hair (˜80 um) are indicated.

FIG. 11 depicts the device used to measure the change in diameter of a lime tree over a two-month period.

FIG. 12A depicts a perspective view of a dendrometer for measuring the diameters of vines and other small-diameter stems.

FIG. 12B depicts a perspective internal view of a dendrometer for measuring the diameters of vines and other small-diameter stems.

FIG. 12C depicts a cross-sectional view of a dendrometer for measuring the diameters of vines and other small-diameter stems.

FIG. 12D depicts a cross-sectional view of a dendrometer for measuring the diameters of vines and other small-diameter stems.

FIG. 12E depicts a perspective view of a dendrometer for measuring the diameters of vines and other small-diameter stems.

FIG. 12F depicts a perspective view of a dendrometer for measuring the diameters of vines and other small-diameter stems.

FIG. 12G depicts a perspective view of a dendrometer for measuring the diameters of vines and other small-diameter stems.

FIG. 12H depicts a cross-sectional view of a dendrometer for measuring the diameters of vines and other small-diameter stems.

FIG. 12I depicts a perspective view of a dendrometer for measuring the diameters of vines and other small-diameter stems.

FIG. 12J depicts a perspective view of a dendrometer for measuring the diameters of vines and other small-diameter stems.

FIG. 12K depicts a perspective view of a dendrometer for measuring the diameters of vines and other small-diameter stems.

FIG. 12L depicts a perspective view of a dendrometer for measuring the diameters of vines and other small-diameter stems.

FIG. 12M depicts a perspective view of a dendrometer for measuring the diameters of vines and other small-diameter stems.

FIG. 12N shows two dendrometers measuring the diameters of two grape vines.

FIG. 12O shows a close-up view of a dendrometer measuring the diameter of a grape vine.

FIG. 12P shows a perspective view of two dendrometers measuring the diameters of two grape vines.

FIG. 13A shows a perspective view of an integrated tree sensor.

FIG. 13B shows a perspective view of an integrated tree sensor.

FIG. 13C shows a cross-sectional view of an integrated tree sensor.

FIG. 13D shows a cross-sectional view of a plunger for an integrated tree sensor.

FIG. 13E shows a perspective view of an integrated tree sensor.

FIG. 13F shows a cross-sectional view of an integrated tree sensor.

FIG. 13G shows a cross-sectional view of an integrated tree sensor.

FIG. 13H shows a perspective internal view of an integrated tree sensor.

FIG. 13I shows a perspective internal view of an integrated tree sensor.

FIG. 13J shows a perspective internal view of an integrated tree sensor.

FIG. 13K shows a perspective internal view of an integrated tree sensor.

FIG. 13L shows a perspective internal view of an integrated tree sensor.

FIG. 13M shows a perspective internal view of an integrated tree sensor.

FIG. 13N shows a cross-sectional view of an integrated tree sensor.

FIG. 13O shows a cross-sectional internal view of an integrated tree sensor.

FIG. 13P shows perspective views of a gimbal tip for a plunger of an integrated tree sensor.

FIG. 13Q shows a cross-sectional internal view of an integrated tree sensor.

FIGS. 14A-14C show exemplary mounting hardware components for mounting an integrated tree sensor to a tree trunk or other large plant part. FIG. 14A shows a simplified side view of an integrated tree sensor with a captive screw and re-adjustable mount screw. FIG. 14B shows a simplified side view of an integrated tree sensor mounted to a tree trunk using a threaded rod and nuts. FIG. 14C shows a simplified cross-sectional view of an integrated tree sensor mounted to a tree trunk using a longer threaded rod and nuts that can be adjusted over time to account for radial tree growth and re-position the plunger in an appropriate position (e.g., amount of extension).

FIGS. 15A & 15B show exemplary accelerometer data obtained from two

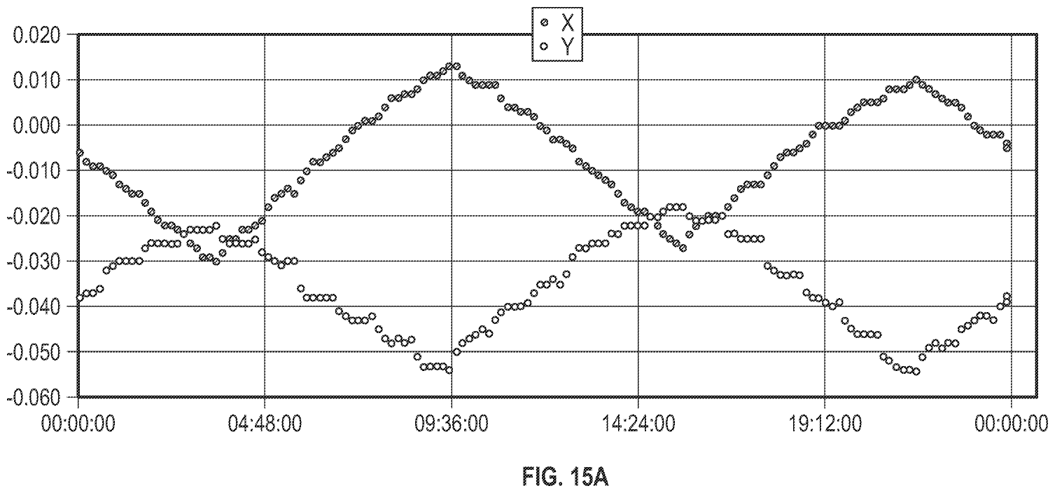

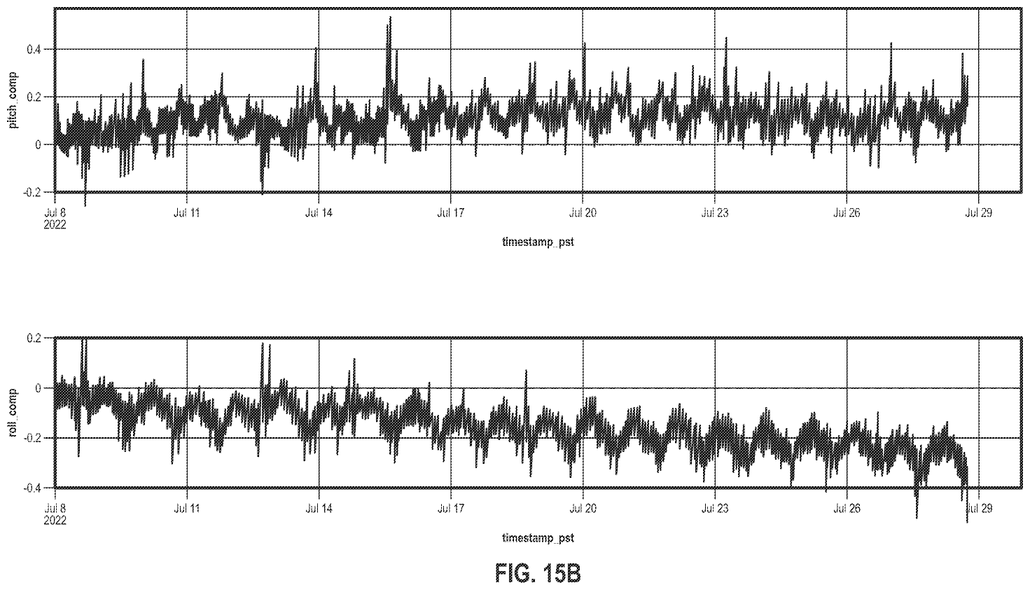

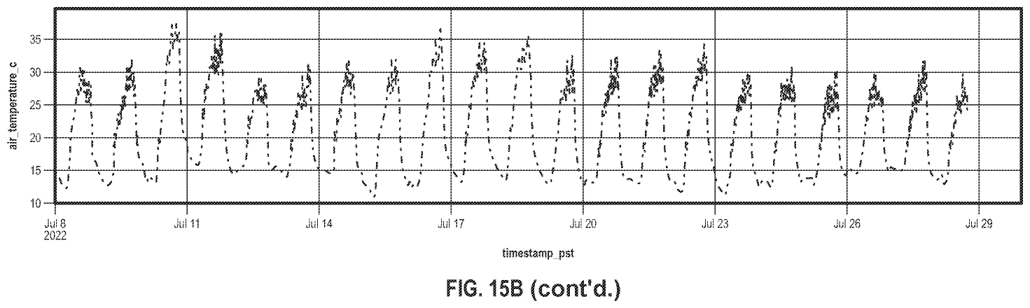

integrated tree sensors mounted next to each other on a leaning part of a citriodora eucalyptus tree. FIG. 15A shows lean over time, with blue dots (top) indicating deviation from the x-axis, and orange dots (bottom) indicating deviation from the y-axis. FIG. 15B shows pitch (top panel), roll (middle panel), and air temperature (bottom panel) measured over time (days).

### DETAILED DESCRIPTION

The following description sets forth exemplary methods, parameters and the like. It should be recognized, however, that such description is not intended as a limitation on the scope of the present disclosure but is instead provided as a description of exemplary embodiments.

### Sensors for Measuring Plant Part Size and/or Other Characteristics

Certain aspects of the present disclosure relate to sensors for measuring plant size (e.g., size of a plant part, such as a stem, bole, shoot, cane, body, branch, vine, trunk, or fruit) and/or other plant part characteristics (e.g., characteristics of the plant part itself or its immediate environment). By collecting data from multiple components integrated within the sensor, the sensors of the present disclosure are thought to allow for richer data sets that can be combined with and cross-validated against each other, thereby providing a more complete picture of the plant than existing devices.

In some embodiments, a sensor of the present disclosure comprises one or more fasteners configured to be positioned in or around a plant part; a processor; a power supply; and two or more components selected from the group consisting of: a dendrometer, an accelerometer, an air temperature sensor, a humidity sensor, and a light sensor. For example, in some embodiments, the sensor comprises a dendrometer and one or more of: an accelerometer, an air temperature sensor, a humidity sensor, and a light sensor. In some embodiments, the sensor comprises a dendrometer, an accelerometer, an air temperature sensor, a humidity sensor, and a light sensor.

In some embodiments, the processor of the sensor comprises a printed circuit board (PCB). In some embodiments, the PCB comprises an epoxy-fiberglass composite material, e.g., in laminated layers (e.g., G10 or FR4). In some embodiments, the PCB comprises a material having stable structural properties and low coefficients of thermal expansion, e.g., as compared to injection molded plastics.

In some embodiments, one or more components of the sensors present disclosure (e.g., a magnetometer, transmitter, solar panel, accelerometer, light sensor, humidity sensor, air temperature sensor, battery, and/or a mount screw or compression-limiting element of the present disclosure) are affixed to the PCB. Thus, in the PCB can act as a structural element in addition to data processing/collection. Plastic parts manufactured by injection molding for high-volume low-cost production suffer from subtle dimensional changes that can occur slowly over time when under load—a time-dependent viscoelastic flow known as creep. Even under very low loads or no loads, irreversible shape changes may occur over time due to sun exposure, material relaxation, humidity and temperature changes. It is therefore desirable for a precision measurement device, particularly one that needs to provide a measurement over long periods of time, to use more stable materials such as aluminum and stainless-steel alloys. Metals are relatively expensive however and are not suitable for enclosures where RF energy must be transmitted or received. Electronics components are commonly mounted on PCBs, which can be made of laminated layers of an epoxy-fiberglass composite material known G10 or FR4. These materials have very stable structural properties and a low coefficient of thermal expansion, especially when compared to injection molded plastics. Therefore using a PCB to support these other components may provide a stable and cost-effective design.

In some embodiments, the power supply of the sensor comprises a battery, a solar panel or cell, or a combination thereof. In some embodiments, the battery is a coin cell battery. In some embodiments, the battery is affixed to the PCB.

In some embodiments, the power supply comprises an integrated solar panel, hybrid capacitor, and lithium battery. In some embodiments, the sensor charges the capacitor/battery during daylight and can operate during multiple days or weeks of operation in darkness on a charged hybrid cap. Since the energy comes from the sun and the amount will vary depending on weather, geographic location, and placement of the device on the plant (or even the possibility of debris or deposits directly contacting the solar panel surface), the device may operate differently depending on energy availability. Higher data collection and transmission rates will be possible when power is high, while the device may moderate both as light and thus power diminishes.

In some embodiments, the sensor further comprises a housing. In certain embodiments, the housing is or comprises plastic or a polymer resin. In some embodiments, the plastic or polymer resin is glass-filled. For example, the plastic or polymer resin can comprise about 10-40% glass, about 20-40% glass, about 30-40% glass, about 10-30% glass, about 15-35% glass, about 25-35% glass, about 10% glass, about 15% glass, about 20% glass, about 25% glass, about 30% glass, about 35% glass, or about 40% glass. In some embodiments, the housing is not an RF shield. In some embodiments, the housing does not comprise an RF-shielding material.

In some embodiments, the rotational sensor, the processor, and/or the power supply are positioned within the housing. In some embodiments, the housing encloses at least the processor and power supply (e.g., battery). In some embodiments, the housing encloses at least the processor and one or more additional component(s). In some embodiments, the housing encloses at least the processor and magnetometer. In some embodiments, the housing is a sealed, overmolded housing comprising an O-ring. For example, a battery of the sensor can be enclosed using a removable lid covering the battery, allowing the rest of the sensor to be sealed in the housing. In some embodiments, the sensor acts as an encapsulated PCA (printer circuit assembly) as the mechanical components for the magnet plunger are all affixed to the PCA. After manufacturing and testing, the entire PCA can be overmolded and hermetically sealed. This protects the electronic components from water and contamination, while other components can be exposed, such as a solar panel, measurement components of a humidity or air temperature sensor, LED, mounting surface, or plunger. In some embodiments, the housing is overmolded as a single piece, i.e., lacking any seals, junctions, or fasteners such as snaps, screws, and the like. In some embodiments, the housing is overmolded as a single piece (i.e., lacking any seals, junctions, or fasteners such as snaps, screws, and the like), and the sensor comprises an integrated solar panel, hybrid capacitor, and lithium battery. Advantageously, this is thought to provide a power source operable for the life of the sensor, allowing a single-piece, overmolded housing to be used (since the housing need not be opened to access and/or replace a battery), thereby providing a permanently and hermetically sealed enclosure for the PCB/PCA and other components. Techniques and sytems for overmolding, including low pressure overmolding, are known in the art; e.g., as used with the Henkel TECHNOMELT® thermoplastic. In some embodiments, the housing comprises a thermoplastic such as Henkel TECHNOMELT® thermoplastic.

In some embodiments, the sensor comprises a dendrometer. In some embodiments, the dendrometer comprises a plunger having a cap and a shaft; a magnet attached to or within the shaft; and a magnetometer configured to detect position of the magnet (e.g., along multiple axes, a radial axis, or a single plane). In some embodiments, the magnet is configured to move laterally in association with the plunger. In some embodiments, the cap is configured to be positioned against the plant part, and the plunger is configured to move laterally (e.g., along multiple axes, a radial axis, or a single plane) in proportion to a change in plant size when the cap is positioned against the plant part. Other dendrometers contemplated for use herein are described infra. Any of the dendrometers of the present disclosure may find use in a sensor as described herein. In some embodiments, the sensor is configured to measure change in diameter or radius of the plant or plant part.

In some embodiments, the magnetometer measures field intensity in two orthogonal axes (e.g., x- and y-axes). As such, the angle of the field lines can be calculated and related to the linear position of the plunger to micron resolution. For example, a ratiometric measurement of the position of the plunger can be used based on the arctangent of the x/y axis position. This differs from a more simple, single-axis magnetometer. In some embodiments, the magnetometer is affixed to a PCB or PCA of the present disclosure.

In some embodiments, the magnet is a rare-earth magnet. In some embodiments, the magnet is a neodymium magnet. In some embodiments, the magnet produces a field characterized by curved field path that changes angle relative to a fixed point as the plunger moves in and out following plant movement. In some embodiments, the magnet is characterized by low changes to field characteristics over the life of the device as long as it is maintained at reasonably low temperatures, i.e. not heated artificially. In some embodiments, the magnet is installed in a plunger assembly that rests on the surface of a tree or woody plant preferably with a very small amount of cork between the plunger and the phloem of the plant which expands and contracts in association with changes in turgor or water potential of the plant. In some embodiments, the magnet is a cylindrical or disc magnet positioned inside the plunger shaft.

In some embodiments, the magnetometer is configured to detect position of the magnet at micron-scale resolution. For example, in some embodiments, the magnetometer is configured to detect position of the magnet at a minimum resolution of at least 1 mm, at least 500 μm, at least 250 μm, at least 100 μm, at least 50 μm, at least 25 μm, at least 10 μm, at least 5 μm, or at least 1 μm. In some embodiments, the magnet generates a magnetic field characterized by curved lines of magnetic flux. In some embodiments, an angle of the magnetic field may be determined based on the intensity of the magnetic field along the at least two axes (e.g., along multiple axes, a radial axis, or a single plane) that is detected by the magnetometer. In some embodiments, the angle may be equal to or related to the arctangent of the magnetic field intensity along a first axis divided by the magnetic field intensity along a second axis. If the sensor is affixed to a plant part, and the diameter of said plant part expands or contracts, the angle of the magnetic field generated by the magnet may change. The change in the angle of the magnetic field may be related to the linear change in the diameter of the plant part. In some embodiments, linear change in the diameter of the plant part may be approximately linearly related to the change in angle of the magnetic field. In some embodiments, the linear change in the diameter may be related to the change in angle of the magnetic field by a seventh-order polynomial. In some embodiments, the linear change in diameter may be related to the change in angle of the magnetic field by a seventh-order polynomial during calibration of the sensor.

In some embodiments, the sensor is configured to provide real-time measurements of the plant or plant part of the present disclosure. In some embodiments, the sensor is configured to measure plant part size multiple times per day. In some embodiments, the sensor is configured to measure plant part size at an interval of 3 hours, 2 hours, 1 hour, 30 minutes, 15 minutes, 10 minutes, 5 minutes, 1 minute, 45 seconds, 30 seconds, 15 seconds, or 5 seconds. In some embodiments, the sensor is configured to measure plant part size at an interval of 5 seconds to 1 hour, 5 seconds to 15 minutes, 5 seconds to 5 minutes, 5 seconds to 1 minute, 1 minute to 1 hour, 1 minute to 30 minutes, 1 minute to 15 minutes, 10 minutes to 1 hour, or 10 minutes to 30 minutes.

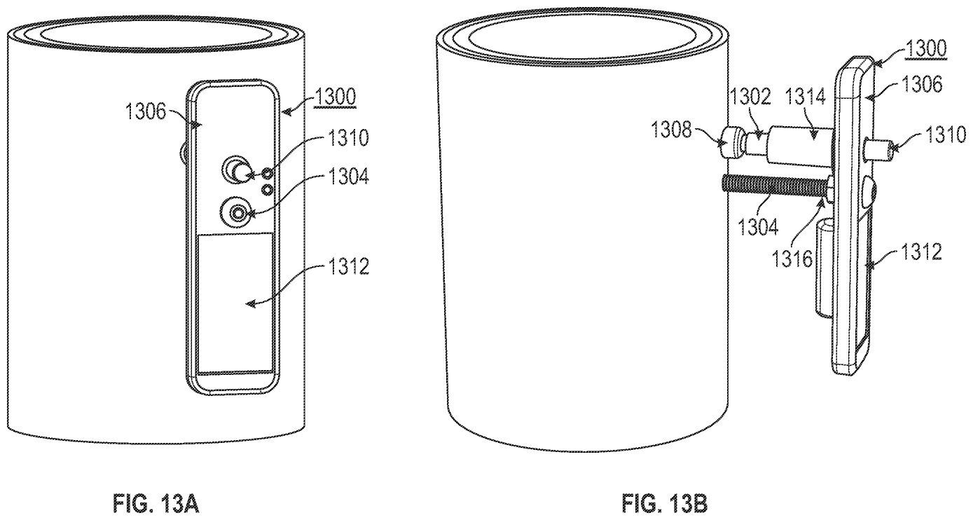

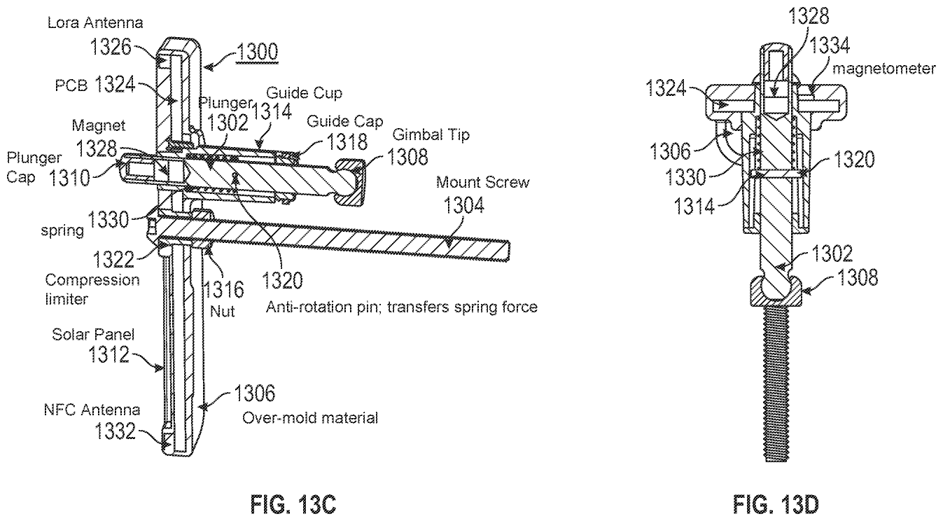

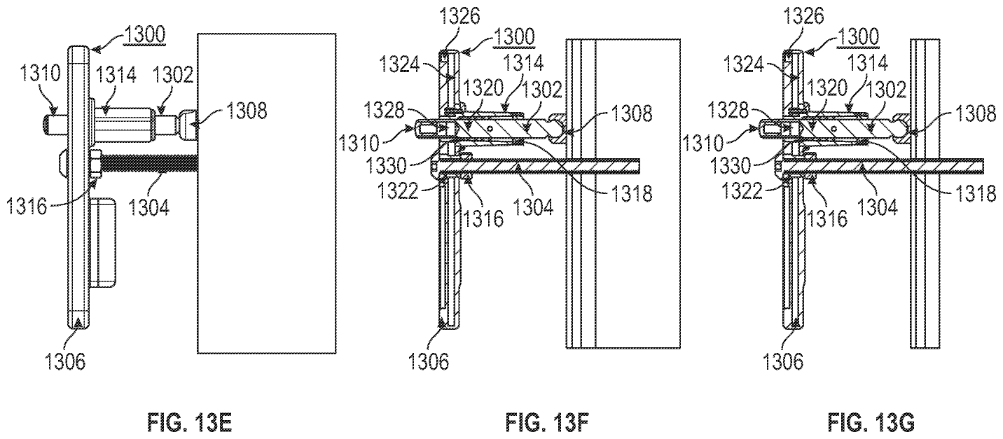

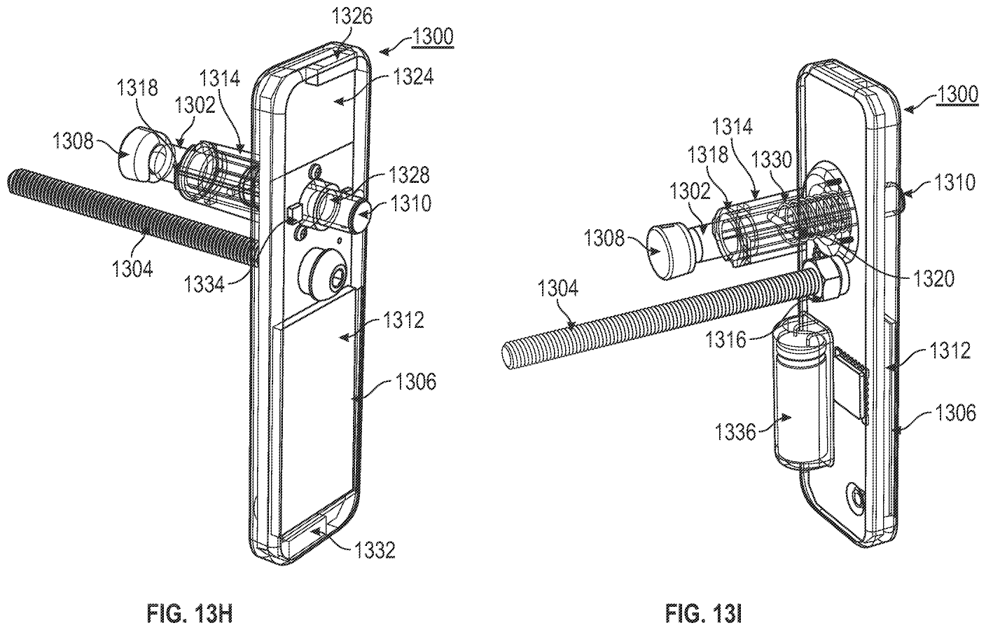

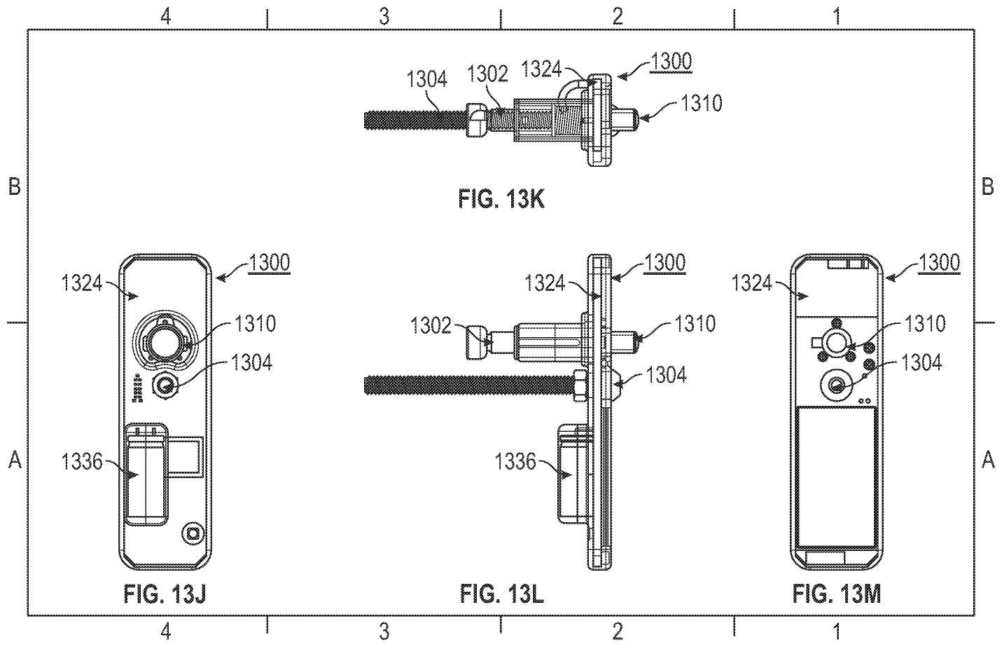

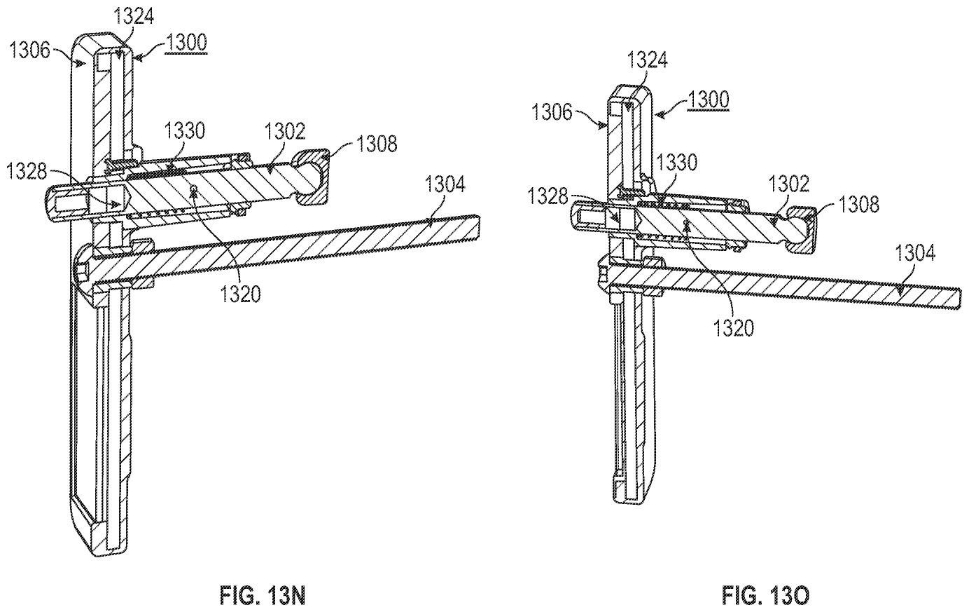

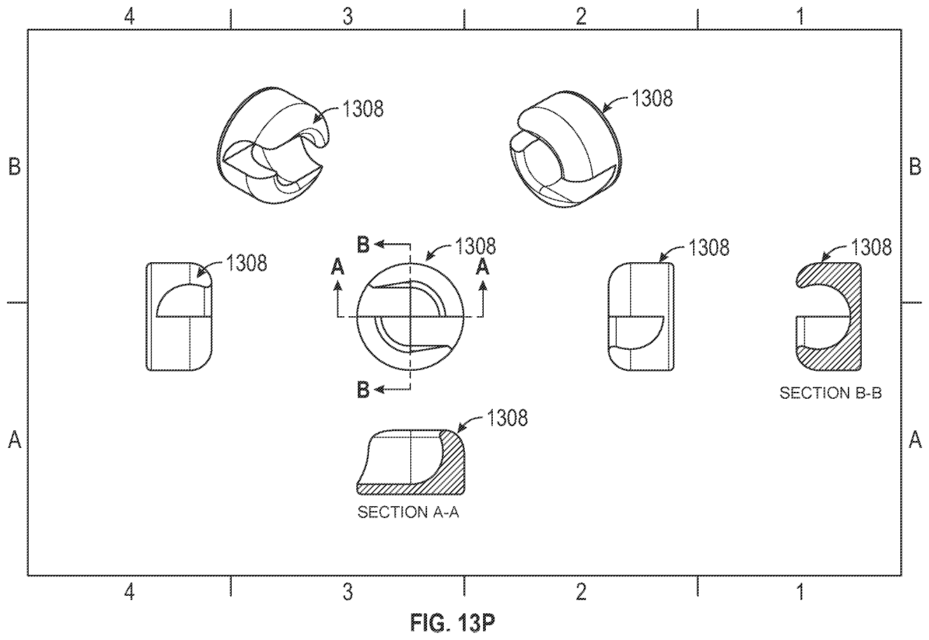

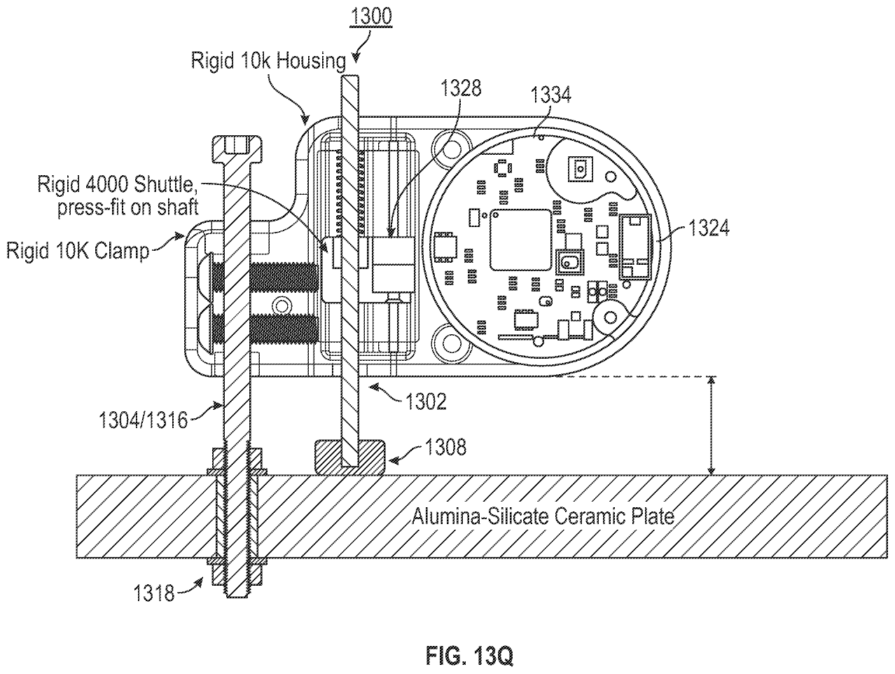

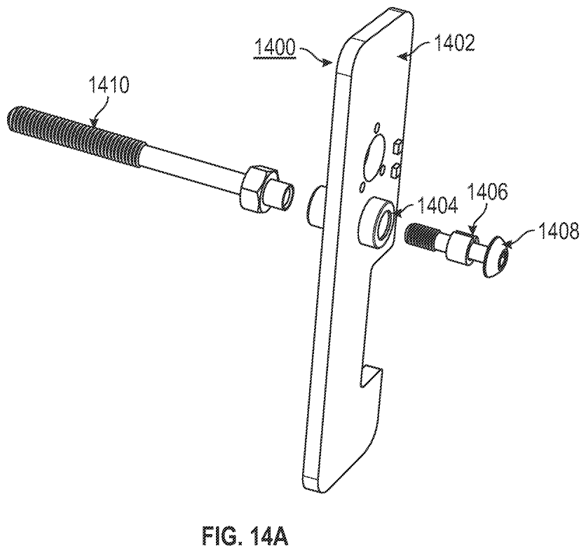

A variety of fastener(s) are contemplated for use in the sensors of the present disclosure, and a person having ordinary skill in the art can suitably select a fastener type based on, e.g., the type of plant part to be measured. In some embodiments, the one or more fastener(s) can include a screw, threaded rod, or nail. The fastener(s) can be configured to be positioned within or onto the plant part and mount the sensor to the plant part. The screw, threaded rod, or nail can be made from a variety of materials, including but not limited to stainless steel, brass, aluminum, or titanium. In some embodiments, a screw can be used to mount the sensor onto the plant part (e.g., a woody branch or trunk) in combination with one or more nut(s), such as a nut configured to be positioned around the screw between the sensor body and the plant part (e.g., nut 1316 in FIGS. 13C & 13Q), and/or a nut configured to be positioned around the screw adjacent to the sensor body but distal to the plant part. In some embodiments, the screw is affixed to a PCB/PCA of the present disclosure.

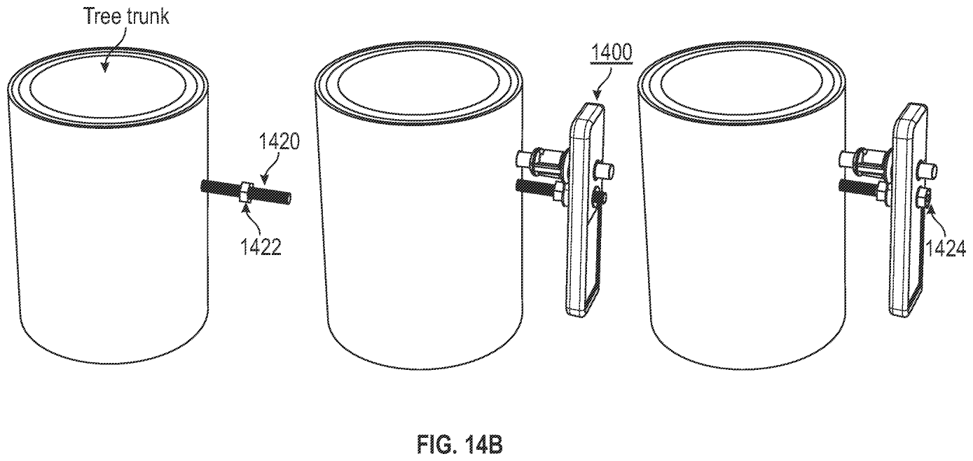

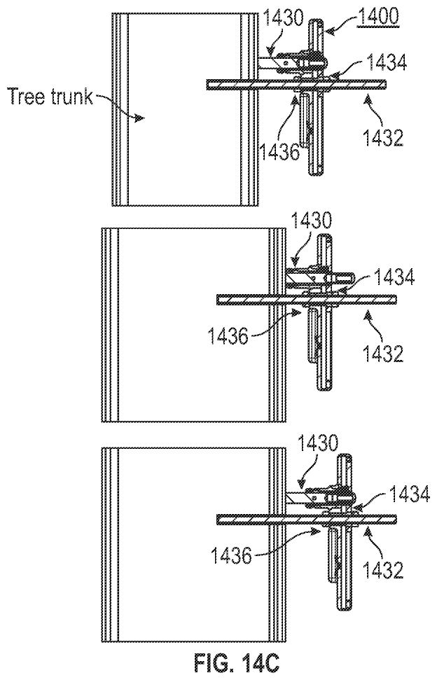

In some embodiments, the sensor further comprises a compression-limiting element. In some embodiments, the compression-limiting element can provide a durable interface between the fastener (e.g., a mount screw) and the rest of the sensor. For example, a compression limiter can be installed in a PCB/PCA of the present disclosure to provide an interface between the PCB/PCA and a fastener such as a mount screw (see, e.g., compression limiter 1322 in FIG. 13C or compression limiter 1404 in FIG. 14A). In some embodiments, the fastener (e.g., a screw) passes through the compression-limiting element. In some embodiments, the compression-limiting element is configured to be positioned around the fastener (e.g., a screw). In some embodiments, the compression-limiting element comprises metal (e.g., a metal collar) or plastic (e.g., a plastic ring). In some embodiments, the compression-limiting element is a ring, O-ring, collar, or washer. In some embodiments, a compression-limiting element is used in combination with a captive screw such that a mount screw (e.g., mount screw 1410 in FIG. 14A) is positioned in the plant part to mount the sensor, one end of the compression-limiting element (e.g., compression limiter 1404 in FIG. 14A) is configured to receive the mount screw (e.g., at an end opposite the end secured in the plant part), and the other end of the compression-limiting element is configured to receive a captive screw (e.g., captive screw 1408 in FIG. 14A). In some embodiments, the captive screw has a button head with a hex socket. In some embodiments, the captive screw is knurled or flanged. In some embodiments, the captive screw comprises a tamper-resistant drive. In some embodiments, the mount screw has a hexagonal nut flange, where the distal face provides a flat surface that the proximal face of the compression-limiting element rests on. This nut shape enables the mount screw to be inserted into the plant part using a standard nut driver. In some embodiments, the distal end of the mount screw has a cylindrical shaped protrusion to locate the compression-limiting element and female threads to receive the captive screw. In some embodiments, the mount screw has a portion with threads and a portion without threads. For example, the portion without threads can be used to indicate correct installation depth. In some embodiments, the sensor further comprises a retaining ring configured to be positioned around the captive screw.

In some embodiments, the one or more fastener(s) can include a threaded rod. In some embodiments, the threaded rod can be used to mount the sensor onto the plant part (e.g., a woody branch or trunk) in combination with one or more nut(s), such as a nut configured to be positioned around the threaded rod between the sensor body and the plant part (e.g., nut 1422 in FIG. 14B or nut 1436 in FIG. 14C), and/or a nut configured to be positioned around the screw adjacent to the sensor body but distal to the plant part (e.g., nut 1424 in FIG. 14B or nut 1434 in FIG. 14C). In some embodiments, the threaded rod and nut configured to be positioned around the threaded rod between the sensor body and the plant part are fused, comprise a single piece of hardware, or the nut is bonded, brazed, soldered, or welded to the threaded rod. In some embodiments, the nut configured to be positioned around the screw adjacent to the sensor body but distal to the plant part is knurled or tabbed. In some embodiments, both nuts are adjustable, e.g., to allow for adjustment of the sensor in relation to the plant part without disassembly (see, e.g., FIG. 14C).

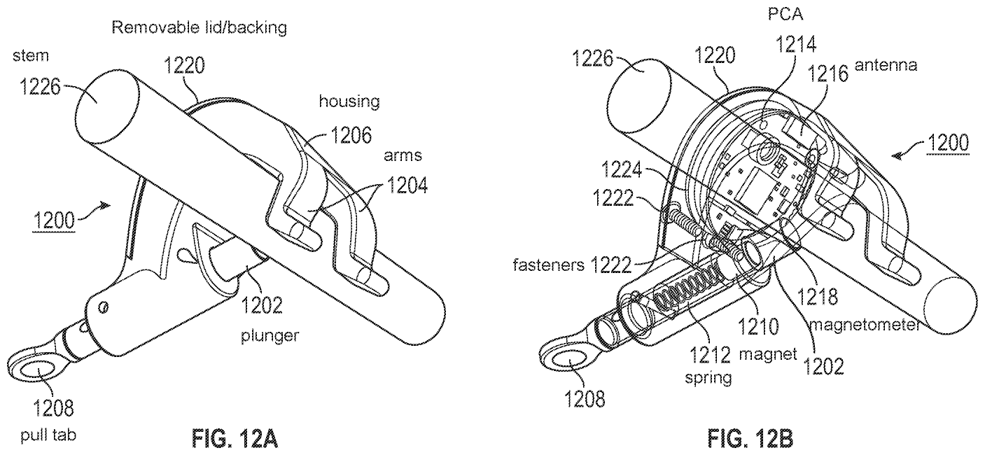

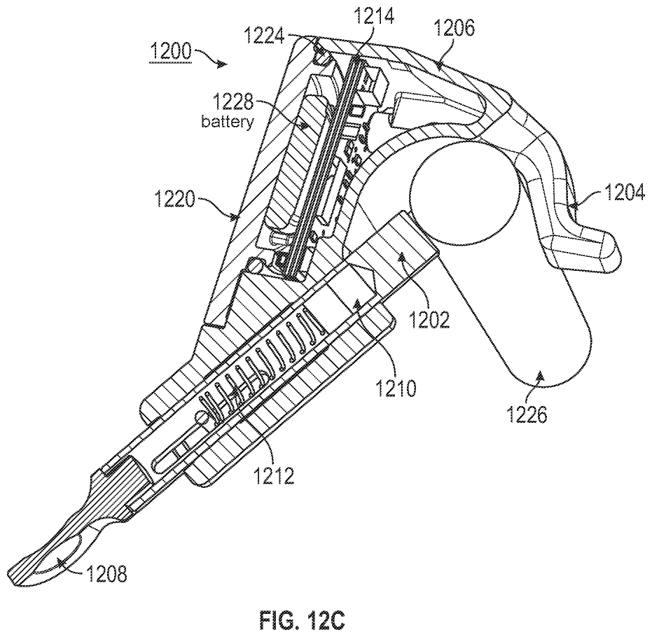

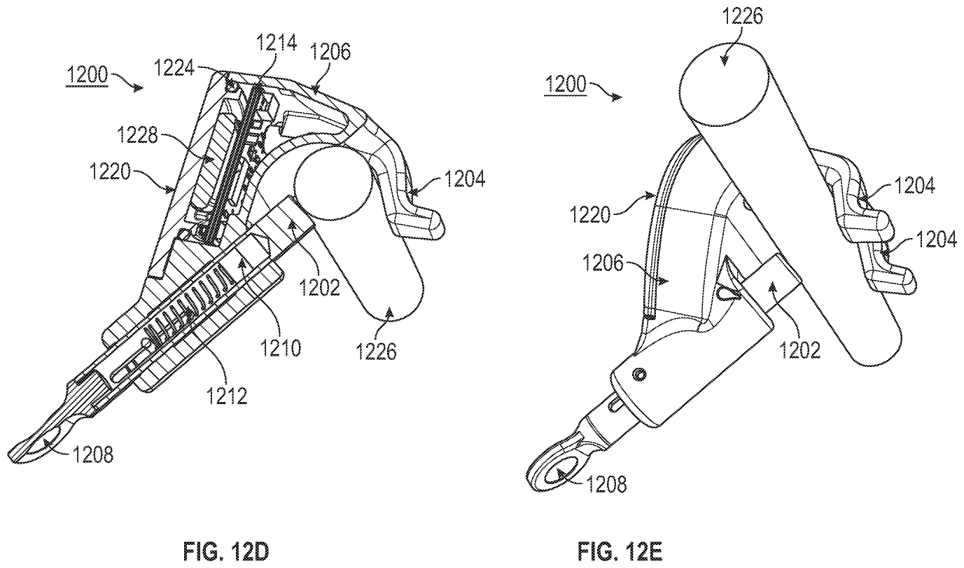

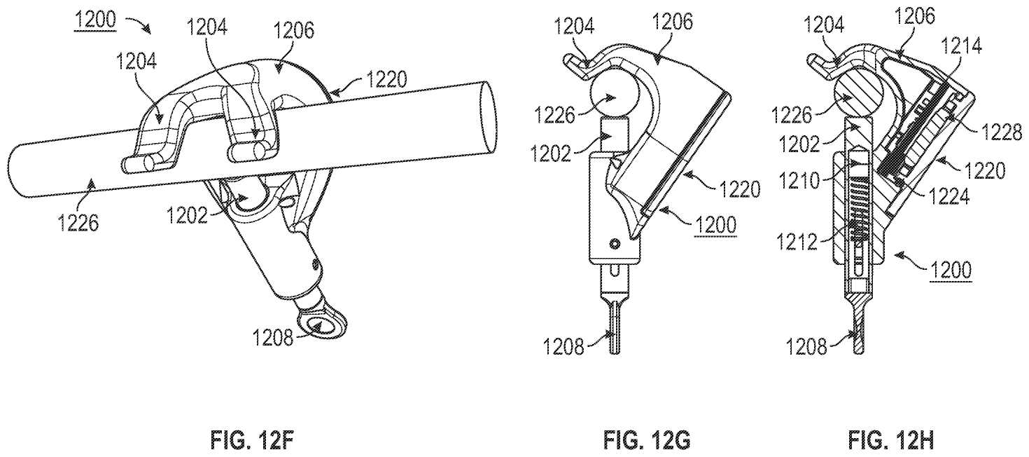

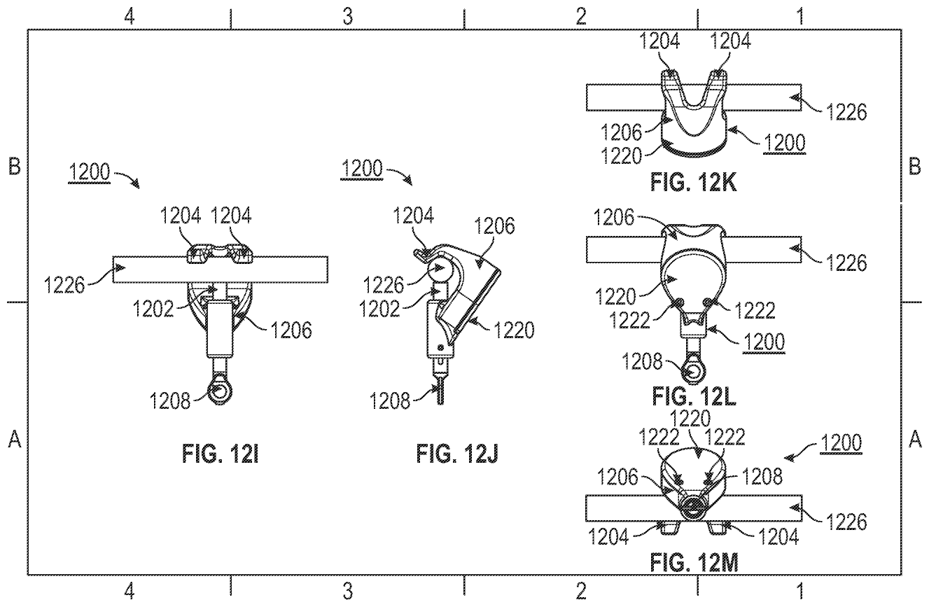

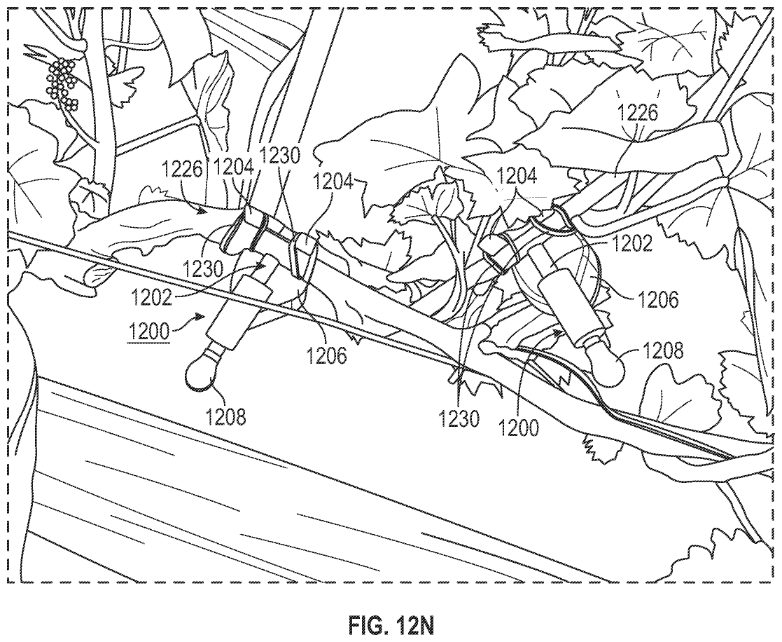

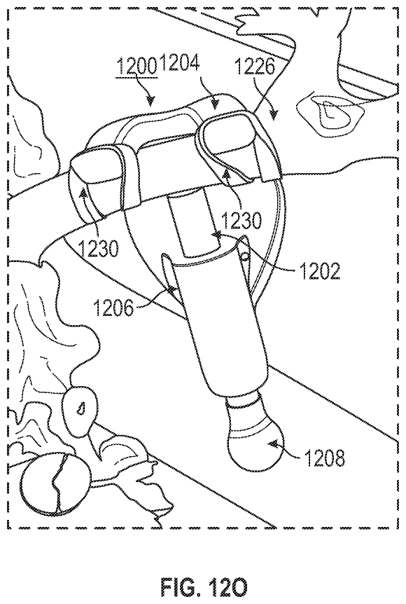

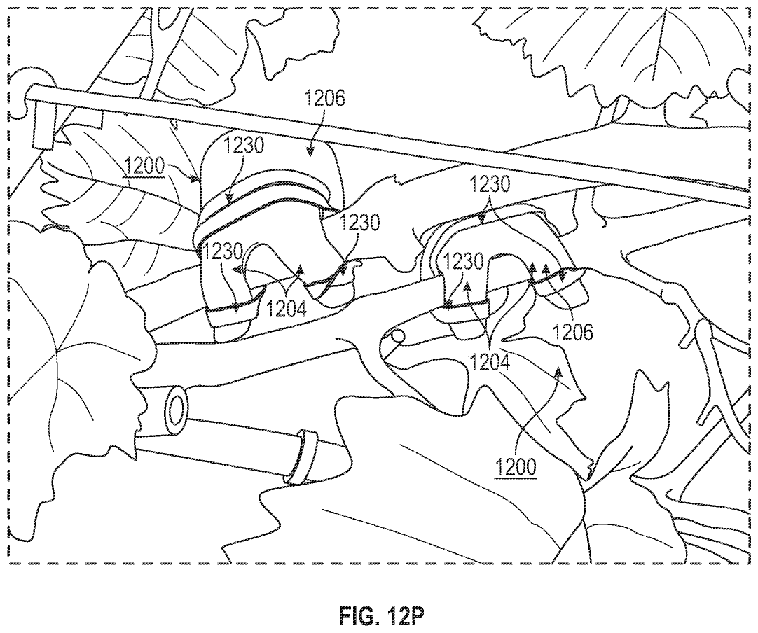

In some embodiments, the fastener(s) can include one or more curved arm(s) configured to be positioned around the plant part. In some embodiments, the fastener(s) can include at least 2, at least 3, at least 4, at least 5, or at least 6 arms. For example, two curved arms arranged in a U- or V-shape can be used, as illustrated in FIGS. 12A-12P. In some embodiments, the one or more curved arm(s) cradles the plant part in a kinematic determinant manner. These embodiments may be particularly useful for smaller plant parts such as stems, shoots, branches, or vines (e.g., grape vines). In some embodiments, the fasterner(s) comprises two or more arms with greater than or equal to 0.15, 0.5, 1, 1.5, 2, or 2.5 inches between arms. These are small and light enough to fit into tight spaces and easy to attach securely to small vines, shoots, stems, and branches. For example, in some embodiments, the vines, shoots, stems, or branches are less than or equal to 0.5, 1, 1.5, 2, 2.5, 3, 3.5, 4, 4.5, or 5 inches in diameter, or greater than or equal to 0.15 inches and less than or equal to 1 inch in diameter.

In some embodiments, one or more elastic band(s) configured to be wrapped around the sensor and the plant part can also be used in combination with the curved arm(s) (see, e.g., elastic band(s) 1230 in FIGS. 12N-12P). In some embodiments, the elastic band(s) is/are resistant to UV radiation.

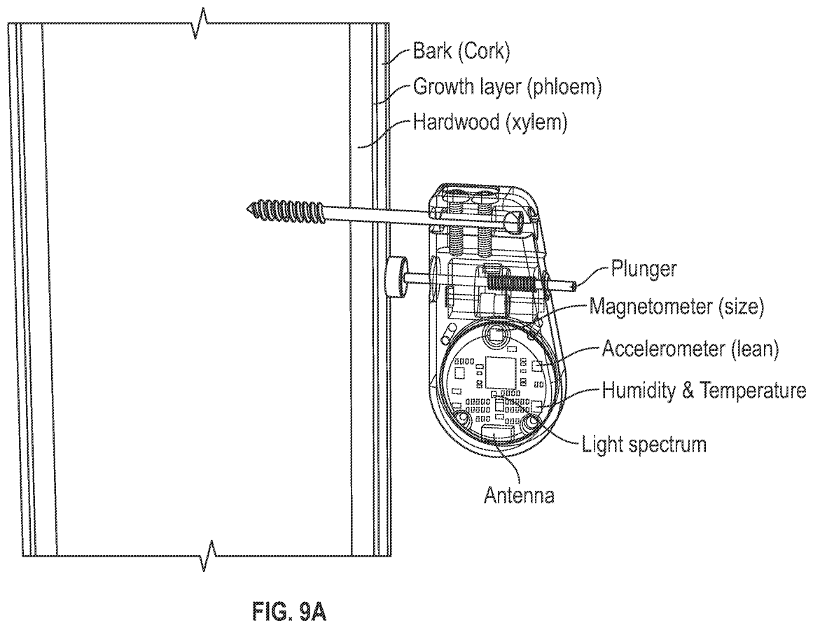

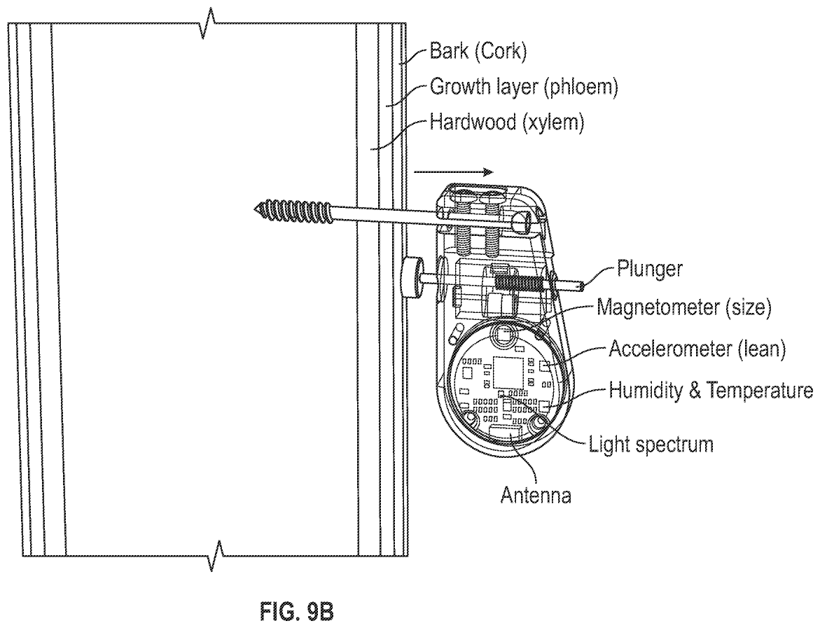

FIGS. 9A & 9B illustrate an exemplary sensor, in accordance with some embodiments. The sensor comprises a plunger, magnetometer (size), accelerometer (lean), antenna, and components that measure humidity, temperature, and light spectrum. The sensor is mounted onto a tree trunk using a mount screw onto which the rest of the sensor is clamped. As the tree grows and its diameter increases, expansion of the phloem pushes the plunger laterally (see arrow in FIG. 9B), and this change in position is monitored by the magnetometer, which detects the position of a magnet affixed to the plunger. In this way, the sensor measures size of the plant part (in this case, a tree trunk). In addition to the magnetometer measuring tree diameter (using the magnet position as a proxy), the light sensor measures sunlight or lack thereof, the temperature sensor measures atmospheric temperature, the humidity sensor measures relative humidity, and the accelerometer measures tree lean (which can be a precursor to tree falling and/or indicate a damaged or unsecured root system).

In some embodiments, the sensor comprises an accelerometer. In some embodiments, the accelerometer is affixed to the PCB. In some embodiments, the accelerometer is a 3-axis accelerometer. In some embodiments, the accelerometer measures lean of the plant or plant part to which the sensor is mounted. In some embodiments, lean as used herein refers to a change in tilt over a timescale, such as days or longer. In some embodiments, the accelerometer measures sway of the plant or plant part to which the sensor is mounted. In some embodiments, sway as used herein refers to movement over a short period of time, e.g., around 1 Hz, or between 0.2 Hz and 20 Hz. In some embodiments, the accelerometer measures impact of the plant or plant part to which the sensor is mounted. In some embodiments, impact as used herein refers to sharp acceleration that may correspond to the plant receiving the force of a collision, e.g., with a vehicle or device. In some embodiments, the accelerometer can be programmed to trigger an alert when a measurement exceeds a predetermined threshold value. For example, the sensor can trigger an alarm when tree lean exceeds a predetermined threshold lean value, indicating that the tree or plant part is at risk of falling.

In some embodiments, the sensor comprises a light sensor. In some embodiments, the light sensor is affixed to the PCB.

In some embodiments, the sensor comprises a humidity sensor. In some embodiments, the humidity sensor is affixed to the PCB. In some embodiments, the housing comprises a port for the humidity sensor to conduct measurements outside of the sensor enclosure. In some embodiments, the humidity sensor measures relative humidity.

In some embodiments, the sensor comprises an air temperature sensor. In some embodiments, the air temperature sensor is affixed to the PCB.

In some embodiments, the sensor further comprises a GPS sensor.

In some embodiments, one or more components of a sensor of the present disclosure can be programmed to trigger an alarm, alert, or other notification when a measurement exceeds a predetermined threshold value. In some embodiments, a processor of the present disclosure can be programmed to trigger an alarm, alert, or other notification when a measurement obtained by one or more components of the sensor exceeds a predetermined threshold value. For example, the sensor can trigger an alarm, alert, or other notification when tree lean exceeds a predetermined threshold lean value based on data from an accelerometer, indicating that the tree or plant part is at risk of falling.

In some embodiments, the sensor of the present disclosure further comprises a transmitter. In some embodiments, the transmitter is a Bluetooth radio or transceiver, e.g., a Bluetooth Low Energy (BLE) radio or transceiver. In some embodiments, the transmitter includes is configured to transmit sensory data wirelessly (e.g., Bluetooth, WiFi, or 900 MHz transmitter) to a mobile device or server. Other possible wireless networks include Narrowband Internet of Things (IoT), LTE-M, and satellite-based networks such as Myriota or Swarm. In some embodiments, the transmitter is a radio. In some embodiments, the transmitter is a transceiver (e.g., Bluetooth transceiver, WiFi transceiver, etc.). In some embodiments, the transmitter is a Long Range (LoRa) transceiver or Near Field Communication (NFC) transceiver. In some embodiments, the transmitter uses Lora radio data transmission system or the LoraWAN network protocol. Advantageously, this provides low-power, long-range transmission. In some embodiments, the transmitter uses a frequency band of about 900 MHz. In some embodiments, the sensor comprises a chip antenna, e.g., the Ignion NN2-2204. In some embodiments, the sensor comprises a split dipole antenna, and two wires extend on opposite sides of the sensor. In some embodiments, the transmitter uses a frequency band of about 900 MHz, and the sensor has a ground plane that is approximately 72 mm (quarter wave length for 900 MHz frequency band) or longer to complement an active antenna side that may be a single wire extending in the opposite direction of the ground plane (up, if the solar panel is down from the mount screw). In some embodiments, the ground plane of the device may be shared with the solar panel.

Advantageously, collecting data from multiple sensors can be used to compensate measurement of diameter change, indirectly compensate to account for mixed signal from bark that could obscure signal from the living plant layers, calibrate and cross-validate data from multiple sources, and understand the drivers of tree growth and/or daily expansion/contraction. For example, these data can be used to approximate and/or predict vapor pressure deficit (VPD) and thereby predict organism dendrometry response. Data can be sent to a server or mobile device via the antenna, creating a distributed IoT network for data collection. These data are high resolution, real-time, and can be collected in a system (e.g., comprising multiple sensors mounted to multiple plants) in which comparisons between multiple organisms can be done (e.g., comparing growth between organisms in similar states, of comparable species, in comparable geographic regions, in comparable weather conditions, in comparable soil conditions, under comparable care/watering/irrigation regimes, etc.). Using these data, model(s) can be constructed for each organism based on observed dendrometry signal, collected environmental or weather data, etc. to predict future dendrometry, e.g., based on current environmental signals or conditions. Further, variance from the model can help to indicate non-measured factors including soil moisture, pests, disease, toxicity, predation, damage, and so forth. As such, it is thought that the sensors of the present disclosure may provide richer data sets and a more complete picture of the plant and its immediate environment than existing sensors (see, e.g., www.phytech.com/home)

In some embodiments, a plunger of the present disclosure comprises a cap and a shaft. In some embodiments, the cap is or comprises molded plastic. In some embodiments, the cap is less than or equal to 5, 4, 3, 2, or 1 mm in thickness. In some embodiments, the cap is configured to contact the plant part over a surface area of between about 10 mm² and about 100 mm², between about 10 mm² and about 50 mm², between about 10 mm² and about 500 mm², or between about 10 mm² and about 1000 mm². In some embodiments, the cap can be molded in low-friction plastic such as acetal or PETG, e.g., using mold side draws. Ideally, the cap makes contact with the plant or plant part over a reasonably-sized area to achieve a consistent measurement and does not apply excessive pressure to the contact area. However, some pressure may be advantageous in maintaining consistent contact with the plant or plant part and/or compressing any minor variations in the cork.

In some embodiments, the cap further comprises a gimbal (e.g., gimbal tip 1308 in FIGS. 13A & 13B). In some embodiments, the gimbal is made with a spherical ball point machined in tree end of the main plunger cylinder that fits in a mating spherical cavity in a tip part that may be injection molded plastic. Advantageously, the gimbal allows the contact surface to comply with the surface of the plant or plant part, e.g., even if the sensor is not mounted in perfect alignment. The gimbal provides some flexibility and tilt that helps to maintain a reasonably-sized contact area; otherwise, the contact area tends to be a small crescent shaped area on the side of the plunger tip that contacts first and the contact pressure will vary over this contact patch with highest pressure at the first point of contact. This introduces a variable that can potentially affect the measurement and produce inconsistent results depending on installation precision.

In some embodiments, the shaft comprises aluminum or stainless steel. In some embodiments, the shaft is a cylinder, and the magnet is a cylindrical magnet positioned inside the plunger shaft. In some embodiments, the cylinder is hollow. In some embodiments, the cylinder comprises aluminum. In some embodiments, the shaft is extendable, e.g., such as a threaded shaft extension. In some embodiments, the shaft is impregnated with PFTE or oil.

In some embodiments, the sensor further comprises a hollow shuttle positioned around the plunger shaft (see, e.g., FIG. 13Q). In some embodiments, the shuttle comprises a resin, such as a glass-filled resin of the present disclosure.

In some embodiments, the sensor further comprises a spring around or affixed to the plunger. In some embodiments, the sensor further comprises a pull tab attached to the plunger shaft, opposite the cap (see, e.g., tab 1208 in FIG. 12A).

In some embodiments, the sensor or housing comprises a removable backing that allows the user to access the PCB/PCA. In some embodiments, the removable backing comprises one or more screws, one or more bolts, and/or one or more rivets.

In some embodiments, the sensor further comprises one or more identifiers. In some embodiments, the sensor further comprises a visual identifier. In certain embodiments, the visual identifier is a QR code or a bar code. In some embodiments, the sensor comprises a radio-frequency identification (RFID) tag.

Advantageously, the sensor of the present disclosure can be used to measure any kind of plant stem, including primary stems, secondary stems, petioles, trunks, reeds, stalks, and the like, as well as any kind of plant bole, shoot, cane, body, branch, vine, trunk, or fruit. It is thought that any plant part susceptible to size fluctuations due to irreversible meristem growth or reversible swelling/contraction as a function of plant hydraulic status or environmental factors (e.g., temperature, relative humidity). The sensor of the present disclosure can be used to measure any type of plant including, but not limited to, vegetables (e.g., tomatoes, etc.), trees (e.g., rubber trees, fruit trees, etc.), row crops, ornamental plants, and the like. In some embodiments, the plant is a crop tree. In some embodiments, the plant is a citrus, olive, nut, cacao, oak, pine, redwood, “strawberry,” or maple tree. In some embodiments, the plant is a woody plant. In some embodiments, the plant is a vine, e.g., a grape vine. Growth of a variety of plants may be monitored with the sensors, systems, and methods disclosed herein.

In some aspects, provided herein is a sensor, comprising: a) one or more fasteners configured to be positioned around a plant part (e.g., a plant stem, body, branch, vine, trunk, or fruit), wherein the one or more fasteners comprise(s) a rotatable element, and the rotatable element is configured to rotate in proportion to a change in plant part size when positioned around the plant part; b) a magnet, wherein the magnet is configured to rotate in accordance with the rotatable element; c) a rotational sensor configured to detect rotation of the magnet; d) a processor; and e) a power supply. Advantageously, these simple and inexpensive sensors are able to provide real-time, rapid, continuous, or near-continuous monitoring of plant growth, which can indicate changes in health, growth, watering, pests, sunlight, temperature, humidity, or other conditions. Such data can be obtained close to the plant or at a distance (e.g., by transmitting data to a mobile device, server, or other computer system) and can easily be adapted for a plurality of plants over a large distance.

In some embodiments, the magnet is configured such that a North-South pole axis of the magnet is perpendicular to a rotational axis of the rotatable element. In some embodiments, the rotational sensor is a Hall sensor. In some embodiments, the Hall sensor is configured to measure movement (e.g., rotation) of the magnet by measuring a sin/cos wave from the magnet or its magnetic field.

In certain embodiments, the Hall sensor is positioned such that a Z-axis of the Hall sensor is parallel with a rotational axis of the rotatable element. In some embodiments, the rotatable element is configured to rotate in proportion to a change in plant part diameter, the plant part radius, the plant part circumference, or a combination thereof, e.g., after installation of the sensor on a plant. In some embodiments, the rotatable element is configured to rotate in one direction in proportion to an increase in plant part diameter, the plant part radius, the plant part circumference, or a combination thereof and rotate in another direction (e.g., an opposite direction) in proportion to a decrease in plant part diameter, the plant part radius, the plant part circumference, or a combination thereof.

In some embodiments, the degree of rotation of the rotatable element is linear relative to plant part size (e.g., diameter, radius, circumference, etc.) by a constant factor. In certain embodiments, the constant factor is about 10 degrees of rotation of the rotatable element per about 1 mm of plant part size change. In certain embodiments, the constant factor is about 5 degrees of rotation of the rotatable element per about 1 mm of plant part size change. In some embodiments, the constant factor is constant over a dynamic range of plant part size. In certain embodiments, the dynamic range of plant part size is from about 4 mm to about 24 mm in diameter. In certain embodiments, the dynamic range of plant part size is from about 4 mm to about 24 mm in diameter, and the constant factor is about 10 degrees of rotation of the rotatable element per about 1 mm of plant part size change. In certain embodiments, the dynamic range of plant part size is from about 4 mm to about 52 mm in diameter. In certain embodiments, the dynamic range of plant part size is from about 4 mm to about 52 mm in diameter, and the dynamic range of plant part size is from about 1 mm to about 5 mm in diameter. In certain embodiments, the dynamic range of plant part size is from about 1 mm to about 5 mm in diameter. In certain embodiments, the dynamic range of plant part size is up to about 5 mm in diameter. In certain embodiments, the dynamic range of plant part size is from about 0.001 mm to about 5 mm in diameter. In certain embodiments, the dynamic range of plant part size is from about 0.001 mm to about 1 mm in diameter. In some embodiments, the constant factor is about 10 degrees of rotation of the rotatable element per about 1 mm of plant part size change, about 9 degrees of rotation of the rotatable element per about 1 mm of plant part size change, about 8 degrees of rotation of the rotatable element per about 1 mm of plant part size change, about 7 degrees of rotation of the rotatable element per about 1 mm of plant part size change, about 6 degrees of rotation of the rotatable element per about 1 mm of plant part size change, about 5 degrees of rotation of the rotatable element per about 1 mm of plant part size change, about 2 degrees of rotation of the rotatable element per about 1 mm of plant part size change, about 1 degree of rotation of the rotatable element per about 1 mm of plant part size change, about 15 degrees of rotation of the rotatable element per about 1 mm of plant part size change, about 20 degrees of rotation of the rotatable element per about 1 mm of plant part size change, or about 25 degrees of rotation of the rotatable element per about 1 mm of plant part size change. In some embodiments, the dynamic range of plant part size is from about 4 mm to about 52 mm in diameter, about 4 mm to about 30 mm in diameter, about 4 mm to about 40 mm in diameter, about 4 mm to about 60 mm in diameter, from about 1 mm to about 52 mm in diameter, about 1 mm to about 30 mm in diameter, about 1 mm to about 40 mm in diameter, about 1mm to about 60 mm in diameter, from about 1 mm to about 10 mm in diameter, about 0.5 mm to about 5 mm in diameter, about 0.1 mm to about 1 mm in diameter, about 0.01 mm to about 1 mm in diameter, about 0.1 mm to about 10 mm in diameter, or about 0.01 mm to about 10 mm in diameter. The skilled artisan will appreciate that the sensors of the present disclosure could be adapted to a range of useful constant factors and/or dynamic ranges.

In some embodiments, a sensor of the present disclosure uses a magnet and Hall sensor system with a single PCB and battery inside injection molded plastic housing, e.g., to produce accurate measurements that can be transmitted via a low power wireless data link. Other sensors and elements are possible, and the low cost of the magnet/Hall sensor pairing make it highly advantageous.

### Clip Type Sensor

In some embodiments of the sensor of the present disclosure, the one or more fasteners comprise(s) at least a first stationary arm having a base and a rotatable arm having a base, wherein the magnet is positioned within the rotatable arm. In some embodiments, a change in size of the plant part causes rotation of the rotatable arm, e.g., to a degree proportional to the change in size (e.g., circumference, diameter, radius, etc.). This type of sensor is referred to herein as a “clip type” or “clip-style” sensor or dendrometer.

In certain embodiments, the one or more fasteners further comprise a second stationary arm. In some embodiments, the stationary arm(s) and rotatable arm are curved. In certain embodiments, the stationary arm(s) and rotatable arm are curved in opposing directions. In some embodiments, the plant part is contacted by three lines of contact, wherein first line is on the first stationary arm, wherein the second line is on the rotatable arm, and wherein the third line is on the sensor opposite the first and/or second line(s), e.g., part of the sensor housing or other component of the sensor other than the arms.

In some embodiments, the clip type sensor further comprises a torsion spring. In some embodiments, the torsion spring is connected to the rotatable arm, to the one of the stationary arms (e.g., the first stationary arm), or a combination thereof. In some embodiments, the torsion spring is connected to the rotatable arm. In certain embodiments, the torsion spring applies torsion to the connection with the sensor, e.g., the housing or other stationary body of the sensor. In some embodiments, the torsion spring is connected to the first stationary arm and the rotatable arm. In certain embodiments, the torsion spring applies torsion to the connection with the rotatable arm. In some embodiments, the base of the rotating arm and the base of the first stationary arm are connected at a hinge comprising the torsion spring.

In some embodiments of the clip type sensor, the rotational sensor is positioned within a housing of the sensor. In other embodiments of the clip type sensor, the rotational sensor is positioned within one of the stationary arms (e.g., within the first stationary arm or within the second stationary arm).

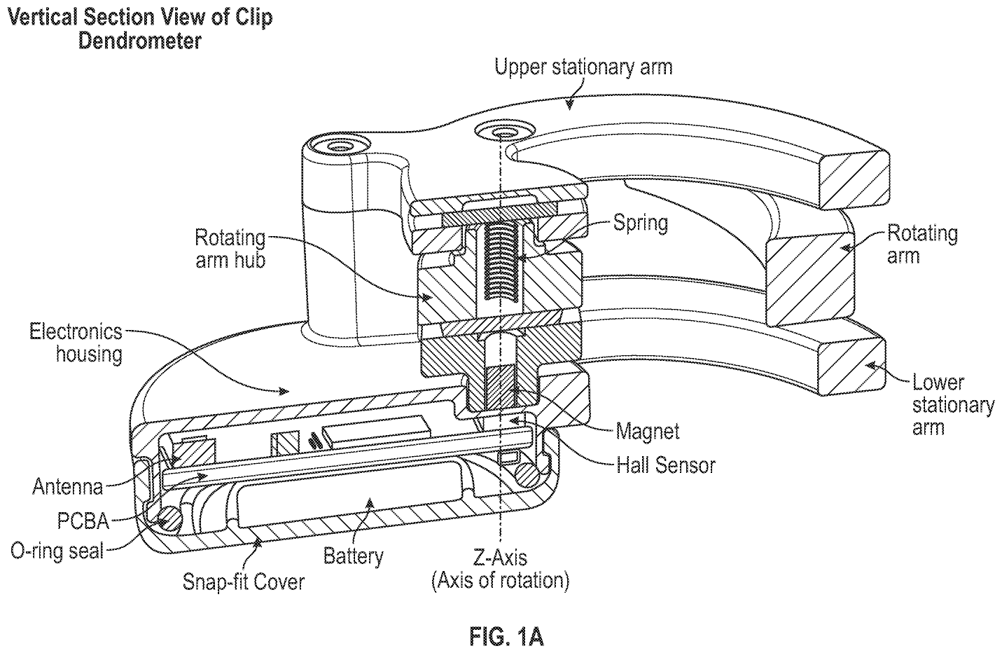

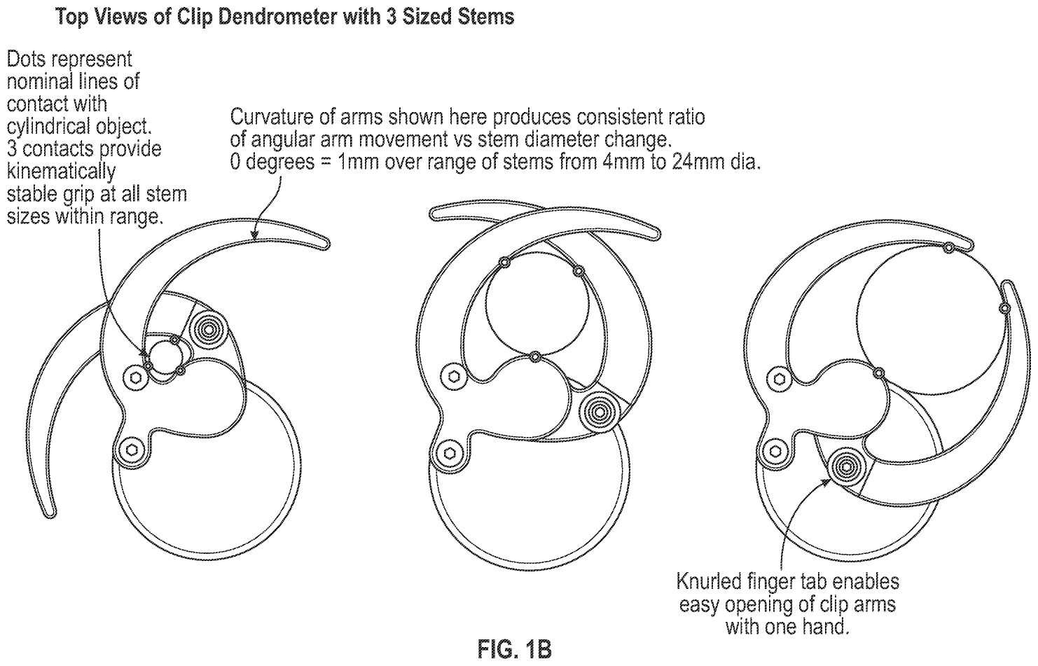

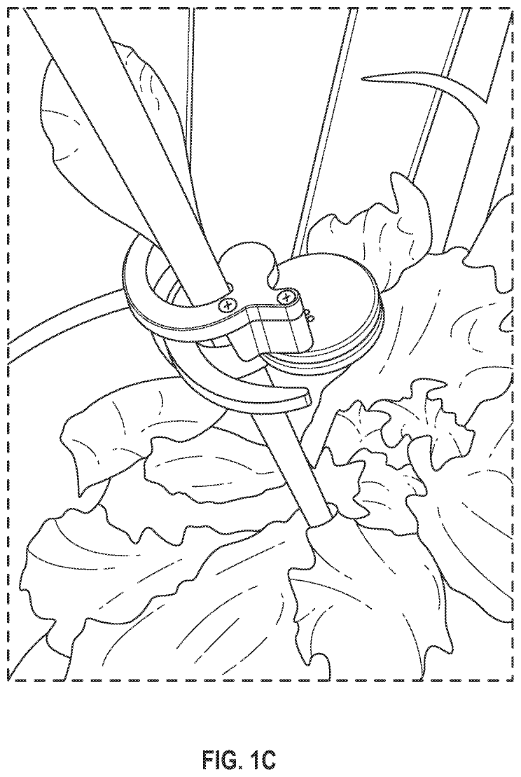

One embodiment of the device includes curved “arms” that are shaped so that a cylindrical object (idealized plant part) is contacted along three lines; one at the body, and one contact on each arm, so that a stable grip of the plant is achieved without any additional restraint. One embodiment of such a configuration is shown in FIGS. 1A & 1B.

In some embodiments, one or more of the arms can be curved such that the angular movement of the measurement arm is linear relative to plant part diameter such as a constant factor, such as 10 degrees of arm rotation per 1 mm of plant part size change. In some embodiments, a magnet is embedded in the arm such that the N-S pole axis is perpendicular to the axis of rotation. In some embodiments, a Hall sensor that can measure field strength in X and Y axes oriented such that the Z axis is aligned with the axis of rotation will detect the rotation of the arms as sine and cosine functions and the angle can be easily calculated as ATAN2 of the X and Y hall signals.

In some embodiments, such devices only include 4 plastic parts, a PCB, a magnet and a spring and can be produced at a very low cost. They are very easy to apply to a plant, requiring only one hand to simply clip in place and begin monitoring. Because the arms both grip and measure the plant, no additional means are required to restrain the system. An exemplary clip type sensor is shown in FIG. 1C.

### Sliding Arm Sensor

In some embodiments of a sensor of the present disclosure (e.g., a clip type sensor), the position of the base of one of the stationary arms is configured to slide relative to the base of the rotational arm, such that sliding the base of the stationary arm a greater distance from the base of the rotational arm causes an increase in the minimum diameter that can be measured by the sensor and a decrease in the minimum change in size that can be measured by the sensor. In other embodiments, the position of the base of the rotational arm is configured to slide relative to the base of one of the stationary arms, such that sliding the base of the rotational arm a greater distance from the base of the stationary arm causes an increase in the minimum diameter that can be measured by the sensor and a decrease in the minimum change in size that can be measured by the sensor. In some embodiments, the position of the base of the first stationary arm is configured to slide relative to the base of the rotational arm, such that sliding the base of the first stationary arm a greater distance from the base of the rotational arm causes an increase in the minimum diameter that can be measured by the sensor and a decrease in the minimum change in size that can be measured by the sensor. In other embodiments, the position of the base of the rotational arm is configured to slide relative to the base of the first stationary arm, such that sliding the base of the rotational arm a greater distance from the base of the first stationary arm causes an increase in the minimum diameter that can be measured by the sensor and a decrease in the minimum change in size that can be measured by the sensor.

The clip type sensor described above is very easy to install and has a good ability to measure the absolute size of anything it is clipped to within its range of measurement. However, most of the time for plant health monitoring the absolute size of the plant part is not as useful to know as the minute changes in size that occur over a short period of time. Measurements taken twice a minute (or similar frequency) for a couple of more of days can show if the plant is expanding and contracting normally for a healthy plant.

A different type of clip sensor in accordance with some embodiments can have a smaller measurement range, such as to detect diameter changes of a maximum of 4 or 10 mm and a higher sensitivity over that range by allowing the arms to slide relative to the measurement portion of the device during installation and then be slid so that the zero rests near the small end of the active measurement range. So the device might be installed on a 30 mm plant part and then the measurement portion set to be at about 1 on a range of 0-5. Now as the plant part grows and contracts over days and weeks it might change from 30 mm to 33 mm with changes as small as 0.001 mm being detected and reported by the device.

### Tape Measure Type Sensor

In some embodiments of the sensor of the present disclosure, the one or more fasteners comprise(s) a clip and a flexible tape with a first end and a second end; wherein the first end is attached to a rotatable drum, wherein the magnet is positioned within the rotatable drum; wherein the second end is configured to be attached with the clip to the sensor; wherein a first section of the flexible tape comprising the first end is configured to be spooled around the rotatable drum; wherein a second section of the flexible tape comprising the second end is configured to be wrapped around the plant part and attached to the sensor with the clip at the second end; and wherein the rotatable drum is configured to rotate in proportion to the change in size of the plant part. This type of sensor is referred to herein as a “tape measure type” or “tape-type” sensor or dendrometer.

In some embodiments, the rotatable drum is configured to rotate in proportion to the change in size of the plant part as the length of the first or second section of the flexible tape changes. In some embodiments, the second section of the flexible tape comprising the second end is configured to be wrapped around the plant part and attached to the sensor at a stationary part or body of the sensor, or a housing of the sensor. In some embodiments, the flexible tape comprises a perforated material, polyethylene terephthalate glycol (PETG), a fluorinated material, a composite material, or any combination thereof. In certain embodiments, the composite material comprises Kevlar, fiberglass, or a combination thereof.

In some embodiments, the tape measure type sensor further comprises a torsion spring; wherein the torsion spring is connected to the rotatable drum; and wherein the torsion spring applies torsion to the rotatable drum or a connection to the sensor thereto. In certain embodiments, the rotational sensor is positioned within a housing of the sensor.

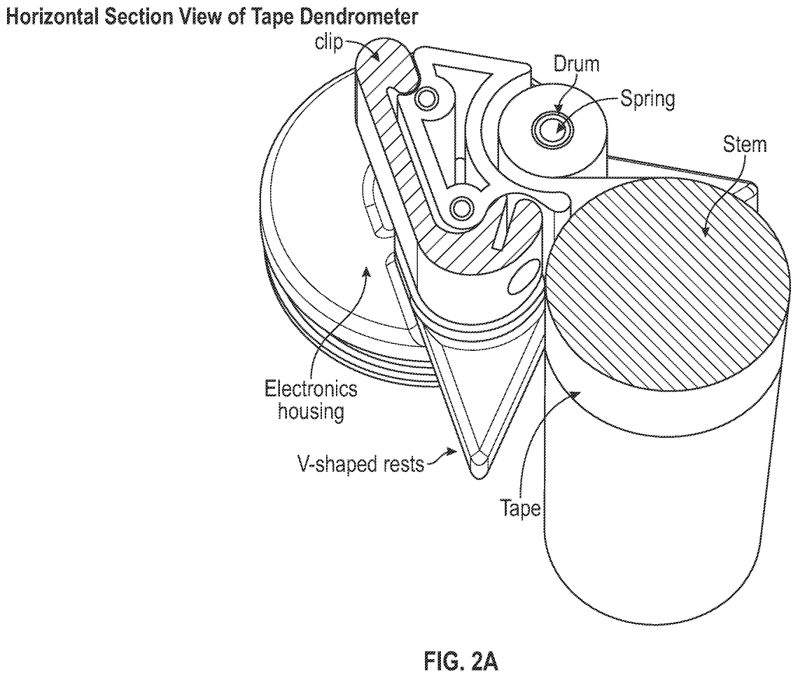

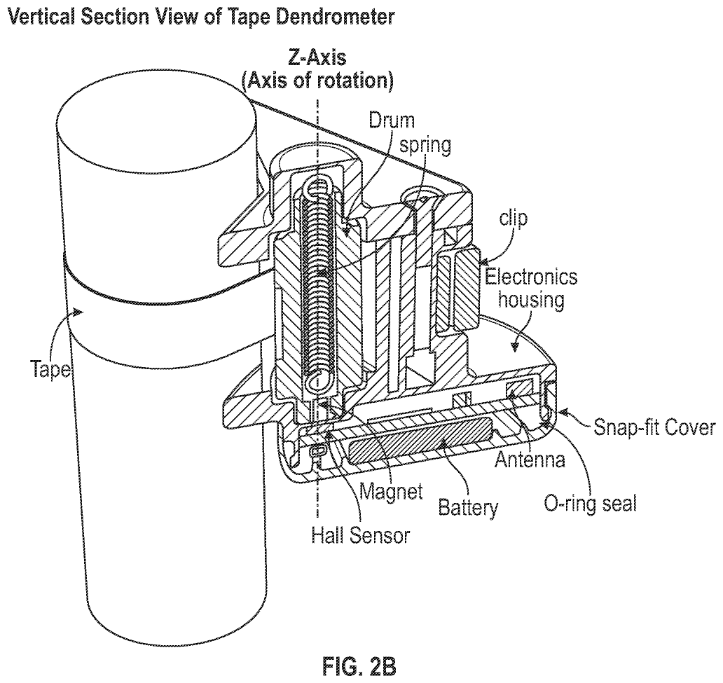

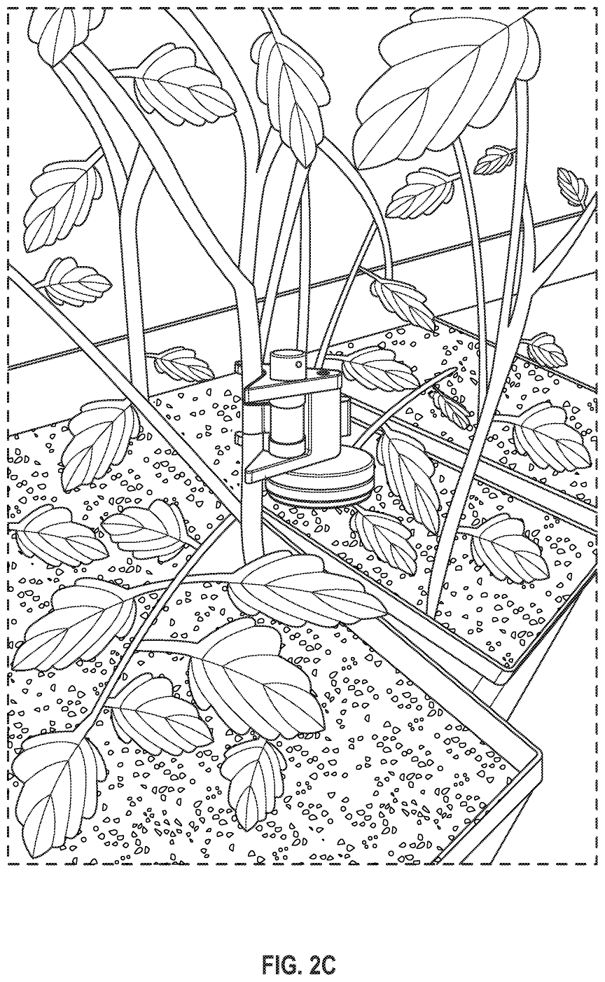

This embodiment of the sensor makes use of a flexible thin band of material that is wrapped around a drum that is restrained with a spring to retract the tape (FIGS. 2A & 2B). The tape is pulled around the plant part and the far end fastened back to the device with a clip. As the plant part increases with size it pulls more tape out and the drum rotates about a Z axis. A magnet is attached in the drum in a similar way to the clip-type sensor described above to produce a measurement signal from the Hall sensor. An exemplary tape measure type sensor is shown in FIG. 2C.

If the drum diameter is relatively small then this device can produce a relatively large measurement signal from a small change in plant part diameter. Also, by including many wraps of tape around the drum a relatively long tape can be included to allow the measurement of larger plant parts.

A potential drawback to this type of sensor vs the clip is that it generally requires two hands to install, it necessarily includes more parts, friction between tape and plant part will reduce measurement fidelity, and the tape could prevent air flow to the plant part. To mitigate these the tape may be made from a perforated material with very low surface energy and low friction. Laser cut PETG is one practical tape selection that works well and is cost effective. Fluorinated materials and composite bands including Kevlar or fiberglass strength elements are also possible.

### Ribbon Type Sensor

In some embodiments of the sensor of the present disclosure, the one or more fasteners comprise(s) a ribbon, a clasp, and a rotatable drum, wherein the magnet is positioned within the rotatable drum; wherein the ribbon is configured to be wrapped around the plant part and fastened to the sensor with the clasp; wherein the rotatable drum is configured to rotate in proportion to the change in change in size of the plant part. This type of sensor is referred to herein as a “ribbon type” or “band-type” sensor or dendrometer.

In some embodiments, the rotatable drum is configured to rotate in proportion to the change in size of the plant part as the position of the ribbon changes. In certain embodiments, the rotational sensor is positioned within a housing of the sensor.

In some embodiments, the sensor further comprises a torsion spring; wherein the torsion spring is connected to the rotatable drum. In some embodiments, the torsion spring applies torsion to the rotatable drum or a connection to the sensor thereto.

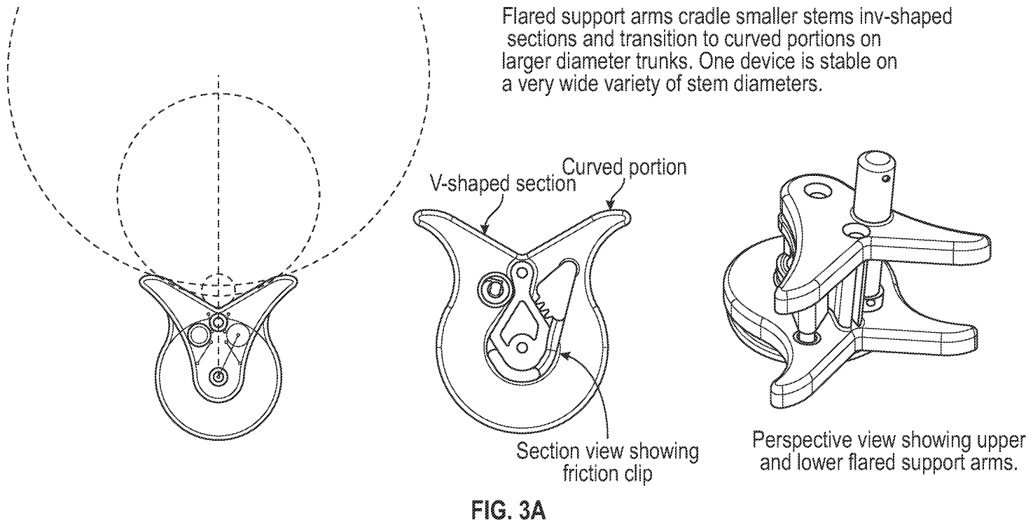

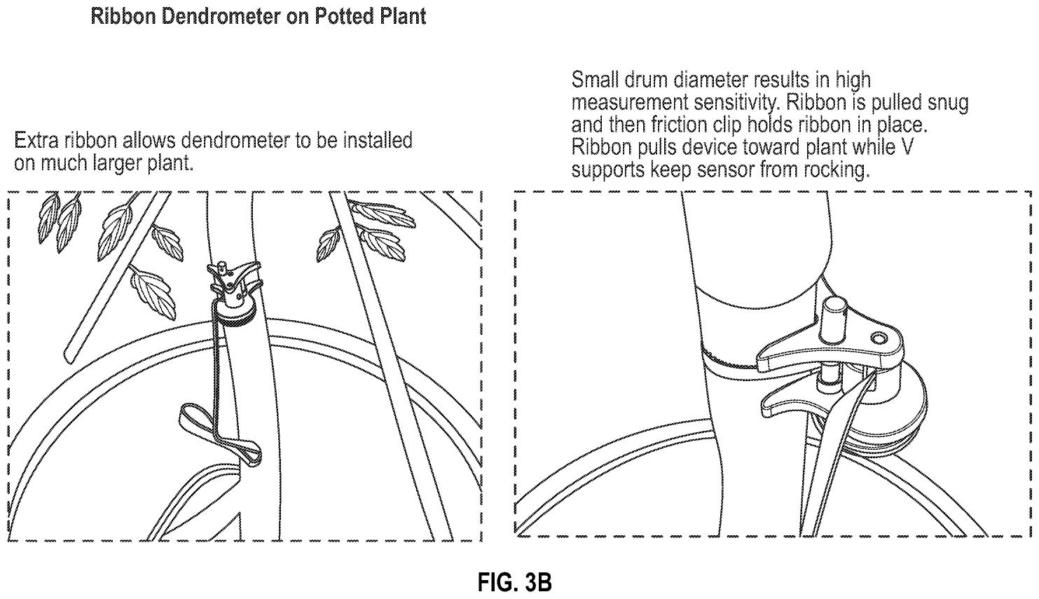

A variant of the tape type sensor has no pre-determined tape length but instead includes a ribbon that can be of arbitrary length to wrap around any size tree (FIGS. 3A-3B). Only the change in ribbon length is measured as generally it is the small changes in plant part (e.g., trunk or stem, etc.) size that are of interest, not the absolute size measurement. The ribbon fastens to the device on the far end via a clasp that grips the ribbon by friction at any point.

### Timing Belt Type Sensor

In some embodiments of the sensor of the present disclosure, the one or more fasteners comprise(s) a belt with a plurality of teeth, a clasp, and a toothed pulley, wherein the magnet is positioned within the toothed pulley; wherein the belt is configured to be wrapped around the plant part and fastened to the sensor with the clasp; wherein the toothed pulley is configured to interlock with one or more of the teeth of the belt and rotate in proportion to the change in size of the plant part. This type of sensor is referred to herein as a “timing belt type sensor”.

In some embodiments, the belt is configured to be wrapped around the plant part and fastened to the sensor with the clasp with teeth facing out, away from plant part. In certain embodiments, the toothed pulley is configured to interlock with one or more of the teeth of the belt and rotate in proportion to the change in size of the plant part as the position of the belt changes.

In some embodiments, the belt comprises Kevlar, metal, fiberglass fibers, or a combination thereof. In certain embodiments, the teeth are spaced about 2 mm apart or less. In certain embodiments, the rotational sensor is positioned within a housing of the sensor.

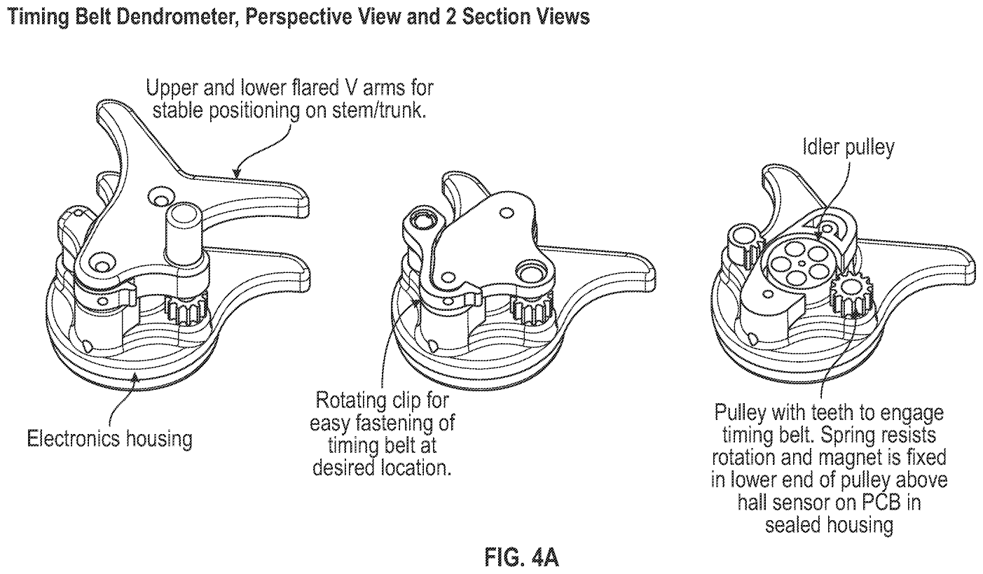

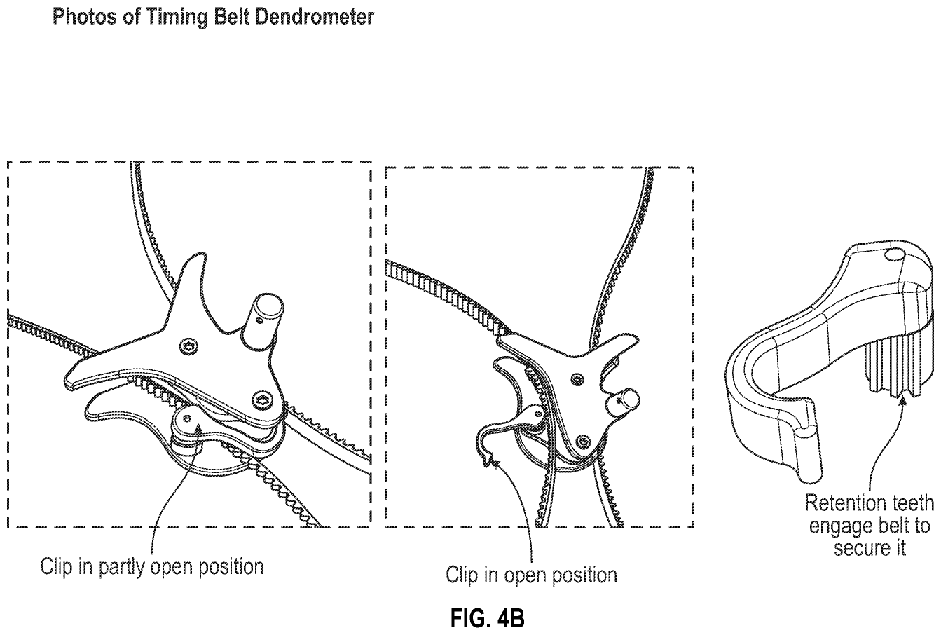

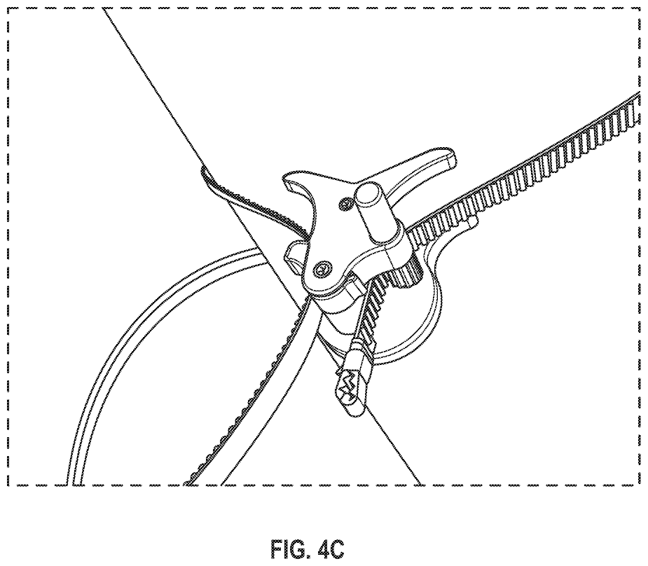

Another embodiment of sensor uses a timing belt so that the teeth side of the belt faces outward when installed around a plant part and the smooth, hard back side of the belt rests against the bark or outer surface of the plant part. The belt can have a hard slippery surface in contact with the surface so as to maximize its ability to slide during the expansion and contraction of the trunk. The Kevlar, metal or fiberglass fibers of the belt resist stretching and thus improve the accuracy of the measurement. Instead of being wrapped around a drum the belt is engaged by a toothed pulley that rotates a magnet so as to produce the measurement (FIG. 4A). The other end can be gripped by a clip on the device at any point. This type also allows measurement of any size plant so long as a timing belt is long enough. 10 m long belts can be easily procured with 2 mm teeth (GT2 profile) for a low price because of their common use by 3D printers. Finer tooth belts custom made for this device could enable even more sensitive measurement of plant parts and offer other benefits, particularly if the inside surface is made of a very hard and slippery surface. An exemplary timing belt type sensor is shown in FIGS. 4A & 4B.

### Systems and Methods for Measuring and Tracking Plant Part Size

In some aspects, provided herein is a system for measuring plant part size and/or other plant part characteristics, comprising: a) a sensor according to any of the embodiments described herein; and b) a mobile device or server; wherein the sensor is connected to the mobile device or server via wireless communication and configured to transmit data to the mobile device or server.

In some embodiments, the sensor is connected to the mobile device or server via Bluetooth low energy (BLE), Long Range (LoRa), or a combination thereof. In some embodiments, the sensor is configured to transmit data to the mobile device or server. In certain embodiments, the sensor is configured to transmit data related to the rotational sensor, plant part size, wireless communication signal strength, or a combination thereof to the mobile device or server. In some embodiments, the sensor is configured to receive data from the mobile device or server.

In some embodiments, the system comprises a plurality of sensors according to any of the embodiments described herein; wherein each sensor of the plurality is connected to the mobile device or server via wireless communication and configured to transmit data to the mobile device or server. In some embodiments, each sensor of the plurality is connected to the mobile device or server via Bluetooth low energy (BLE), Long Range (LoRa), or a combination thereof. In certain embodiments, each sensor in the plurality is configured to transmit data related to wireless communication signal strength to the mobile device or server. In certain embodiments, the mobile device or server receives wireless communication signal strength information from each sensor in the plurality and generates a map of wireless communication signal strength across the locations of the plurality of sensors. In some embodiments, the mobile device comprises a GPS sensor. In certain embodiments, the GPS sensor is configured to obtain location information using the GPS sensor and associate the location information with a sensor of the plurality. In some embodiments, the mobile device comprises a camera or other image sensor (e.g., a CCD or CMOS sensor).

For all dendrometer types described here, a smart phone app can help collect contextual Information using common smart phone sensors (GPS, Compass, RFID, Camera) and question prompts for the user.

Dendrometer measurements are most meaningful if the context is understood well. The type of plant, its location and stage of growth all factor in. Much of this information can be easily captured using a smart phone. The dendrometer devices may have a near field communication device (RFID) that the smart phone will be able to detect and use to identify the device. Alternatively, the device may have a QR code, bar code or other visual identifier that a person or a camera on a smart phone can use to identify the device. One or more pictures taken of the plant the where the device is being installed that will contain information including location from the phone's GPS (Geotag) and the plant may be identifiable by using cloud based plant ID image recognition software. The phone app may prompt the installer to answer a few questions as well, such as if the plant is established or a new planting.

Each device when paired with a smart phone may be used as a network signal strength test device. The device may have two wireless links such as BLE (Bluetooth Low Energy) and LoRa. The LoRa signal may be the primary means of transmitting data from the sensor to the internet system because of its long range and low power consumption while the Bluetooth may be used to directly communicate with the smart phone, since most smart phones support that standard. LoRa signal strength may be measured by the device while it is communicating with the smart phone via BLE. By walking around with the sensor device, or trying out different possible mounting locations—on either side of a tree, for example—the phone may be used to determine the quality of the LoRa communication link at each possible mounting position. This information may be stored as geo-referenced data to map out zones of good signal quality for a given gateway location. A process where by a gateway may be temporarily installed in a trial location and then signal quality is assessed using simply a smart phone and any sensor device that has this two radio feature would make it easier for users to setup a good wireless network for their location and desired sensor placements. For devices with only one radio, such as only LoRa, the same process may apply if the gateway is connected to the internet and the smart phone has network connectivity via cell or wifi. In this case the sensor device is first connected to a gateway when in range and signal quality information is relayed via the internet back end to the phone as the person moves the sensor device around. A real time display on the smart phone screen of signal quality, number of bars and/or color; green good, yellow OK, orange poor, red bad, would enable an installer to easily place sensors in locations that have adequate connectivity. One side of a tree may be sunny, and it is preferable to put the sensor in the shade, but that is less important than having adequate connectivity. On the other hand, yellow connectivity and shade is better than green connectivity and sun. The direction of the sun may be shown using the smart phones app and information about the geolocation. Having both pieces of information on display during install would make it possible for the app to guide and installer to the best sensor placement.

In some aspects, provided herein is a method for tracking plant part size and/or other plant part characteristics, comprising: measuring size and/or other plant part characteristics of the plant part using a sensor of the present disclosure, e.g., based on data collected using its integrated component(s). In some embodiments, the method further comprises measuring size and/or other plant part characteristics of the plant part using a sensor of the present disclosure at a second time after the first time, wherein size and/or other plant part characteristics of the plant part is/are measured using a sensor of the present disclosure, e.g., based on data collected using its integrated component(s). In some embodiments, size and/or other plant part characteristics of the plant part are compared between the first and second times to track changes in the size and/or other plant part characteristics over time (i.e., between the first and second times).

In some embodiments, the size measurement is based at least in part on position of a magnet of the sensor (e.g., as detected by a magnetometer of the present disclosure). In some embodiments, the method comprises, prior to the size measurement, mounting the sensor to the plant or plant part, wherein the one or more fasteners is/are positioned in or around the plant part, and wherein the plunger cap is positioned against the plant part. In some embodiments, the method further comprises measuring size of the plant part using a sensor of the present disclosure at a second time after the first time, wherein the measurement of size at the second time is based at least in part on position of the magnet, and wherein a change in position of the magnet from the first to the second time indicates a change in size of the plant part.

In some aspects, provided herein is a method for tracking plant part size and/or other plant part characteristics, comprising: a) at a first time, measuring plant part size at a sensor according to any of the embodiments described herein; and b) at a second time after the first time, measuring plant part size and/or other plant part characteristics at the sensor, e.g., based on data collected using its integrated component(s). In some embodiments, size and/or other plant part characteristics of the plant part are compared between the first and second times to track changes in the size and/or other plant part characteristics over time (i.e., between the first and second times).

In some embodiments, a change in size of the plant part between the first and second times causes rotation of the rotatable element proportional to the change in size. In some embodiments, a difference in size and/or other plant part characteristics is measured between two timepoints. In some embodiments, a size and/or other plant part characteristic(s) is measured at each time point.

### EXAMPLES

The presently disclosed subject matter will be better understood by reference to the following Examples, which are provided as exemplary of the invention, and not by way of limitation.

### Example 1: Measurement of Plant Stem Size With Dendrometers

Nine dendrometers were installed in an indoor grow room on seven plants and one reference cylinder. Six of the plants were tomatoes, one a rubber plant. On one of the tomatoes, two dendrometers were installed, one above the other on the stem (ed3 and ed4). Seven of the dendrometers were clip-style, and two (TM1 and TM2) were band-style. The grow lights were activated from 5:30 am to 7 pm local (Pacific) time. Watering events were recorded.

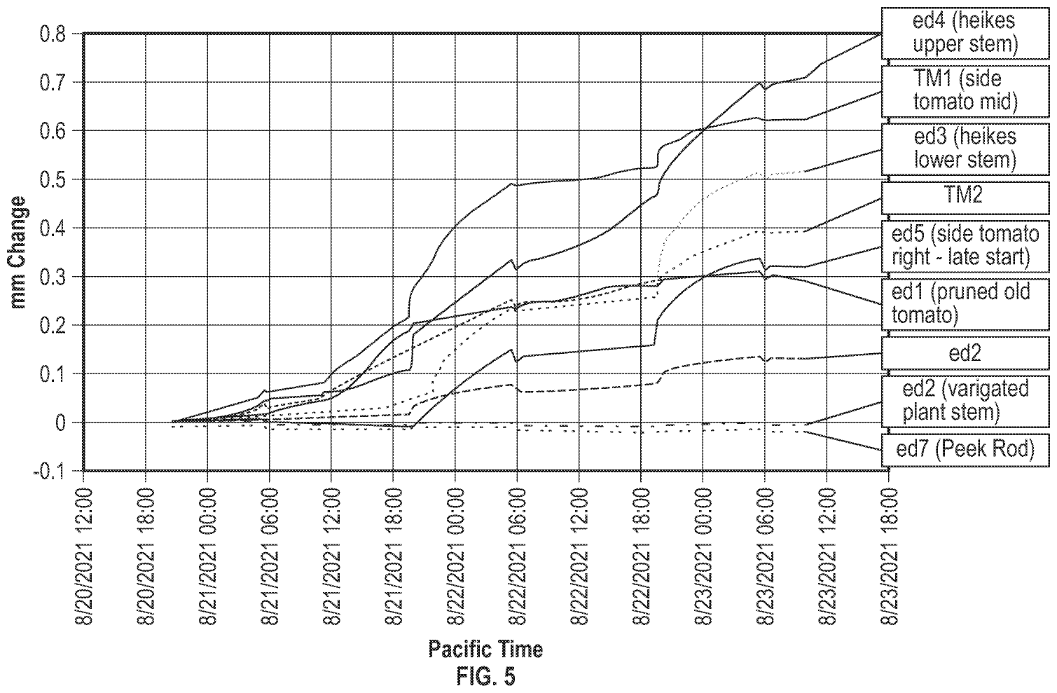

FIG. 5 shows a plot of the stem size measurements recorded by each dendrometer over time. The diurnal cycle can be observed, as can the watering events. A daily disruption correlated with lighting shifts and some amount of settling can be observed on the reference rod. This is expected on these devices and can be corrected.

### Example 2: Measurement of Fuyu Persimmon Tree Stem Size

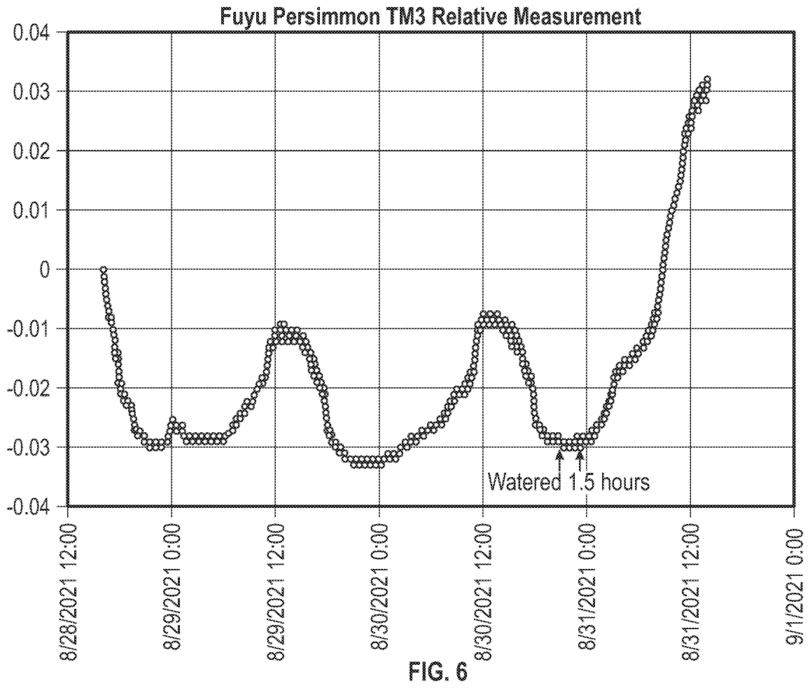

A Fuyu Persimmon tree was in relatively dry soil. FIG. 6 shows data from a tape measure type dendrometer applied to the tree which reported measurements every 30 seconds via bluetooth through a rooftop gateway to cloud based data storage. Measurements were in mm and time is shown as local, pacific time. A daily cycle of approximately 0.02 mm was observed in the size measurement. Water was provided on the third evening in this study period. The following day the stem size increased by about 0.06 mm from the minimum.

### Example 3: Measurement of Tree Size, Air Temperature, Relative Humidity, Magnetometer Temperature, Battery Level, Light Intensity, and Accelerometer Axes

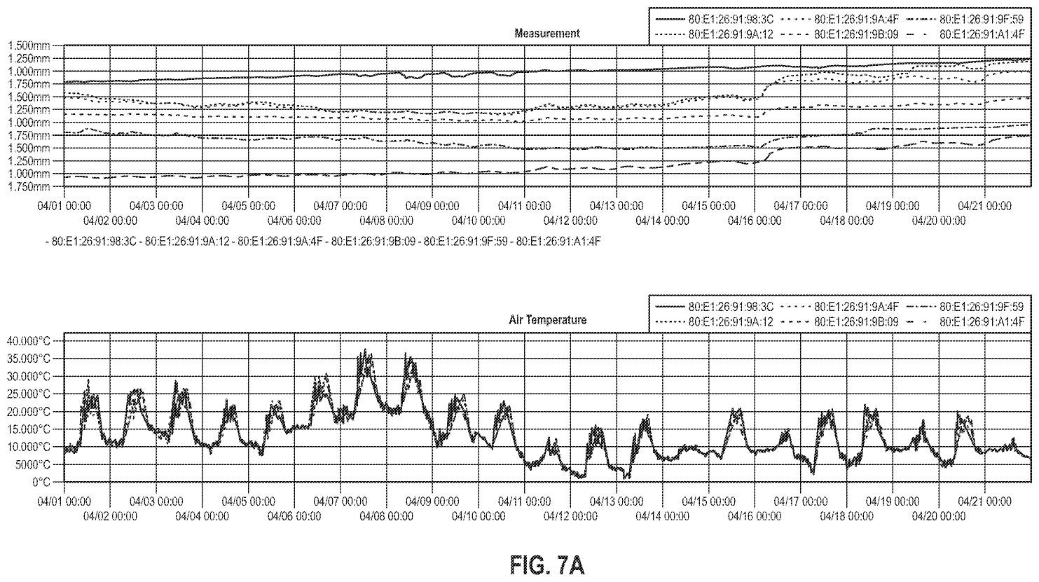

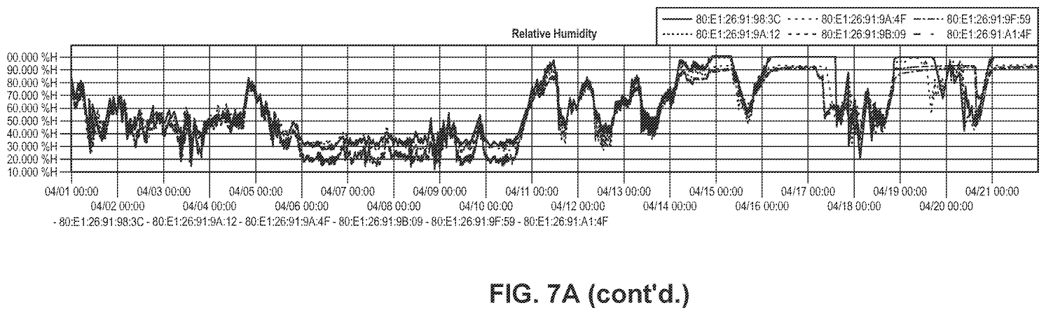

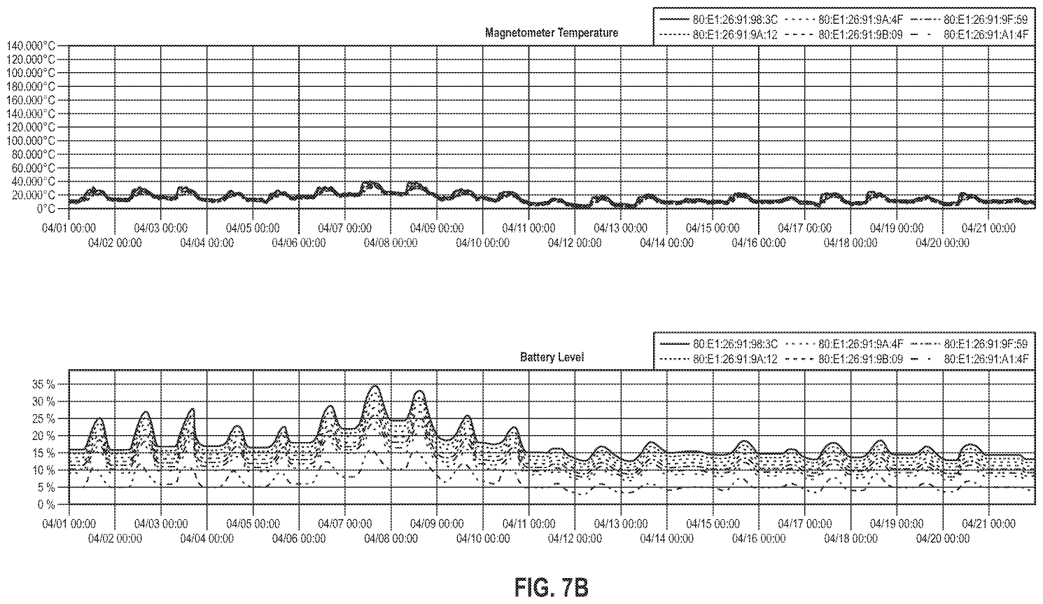

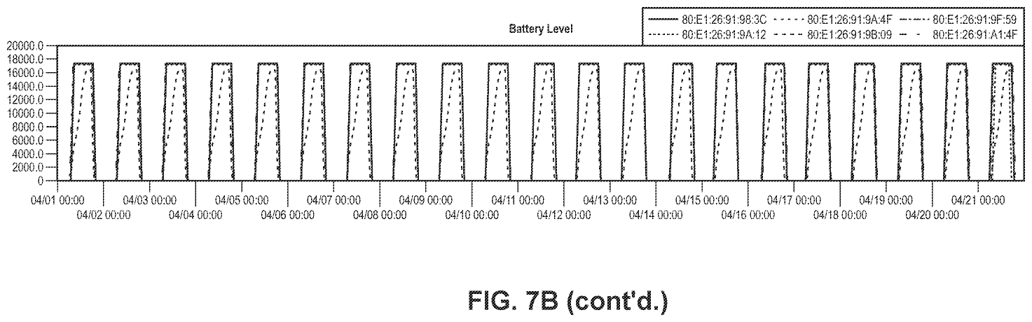

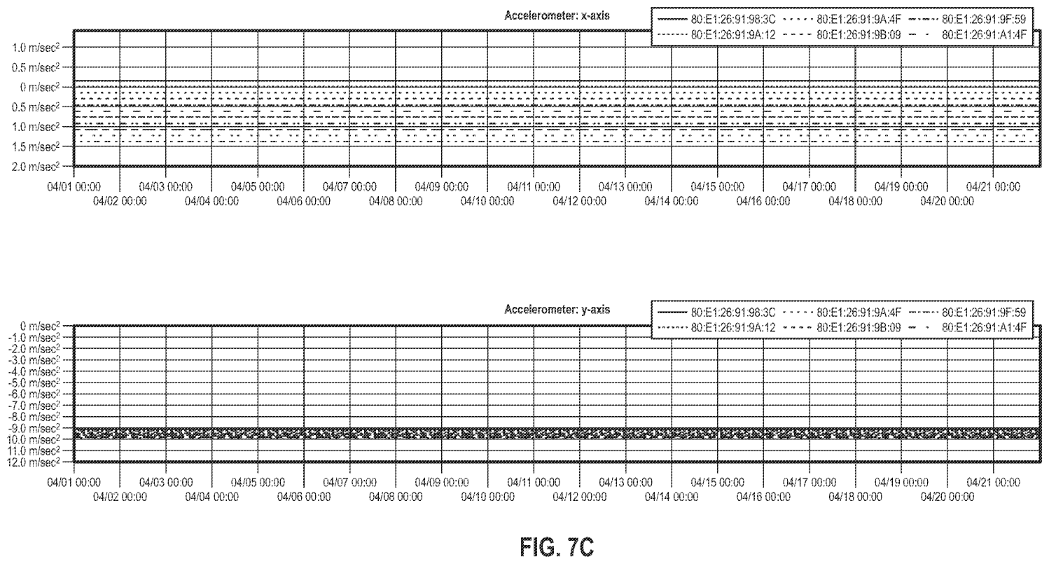

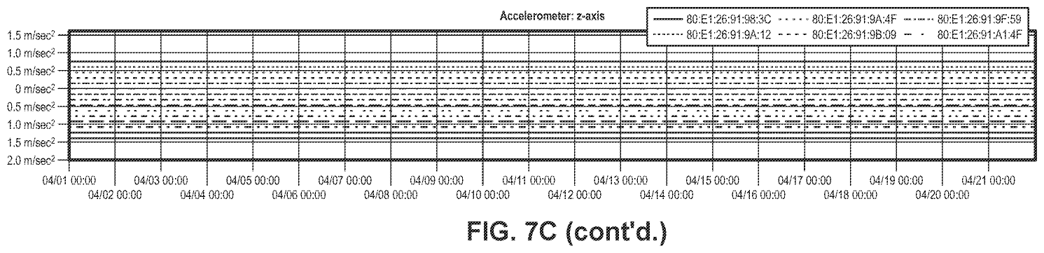

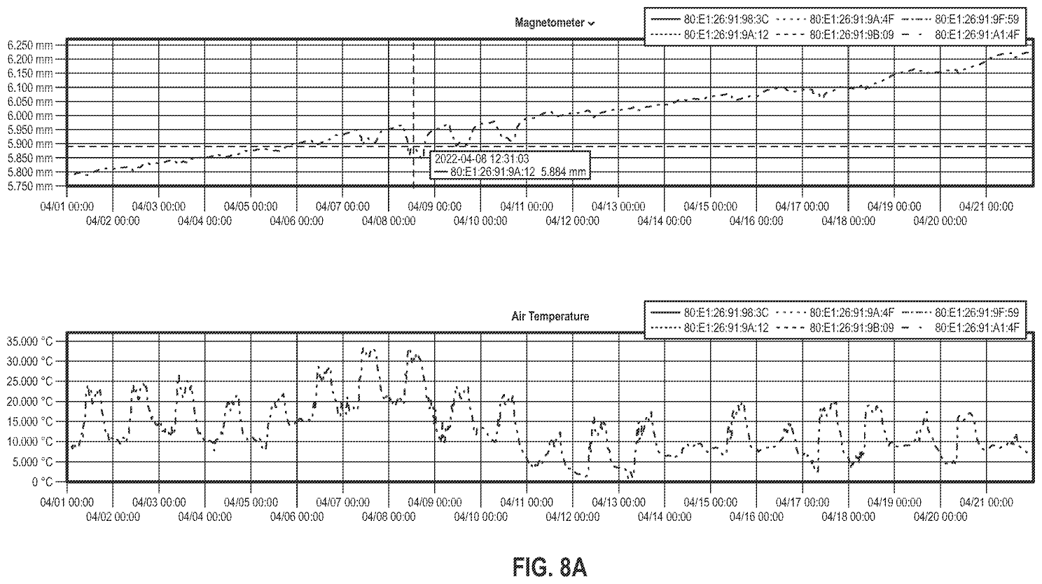

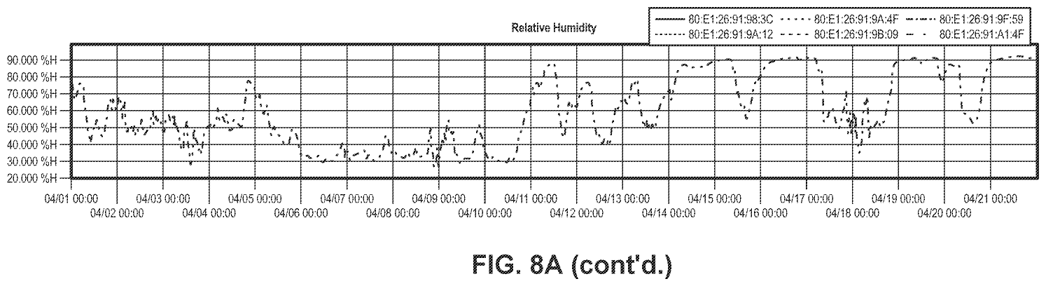

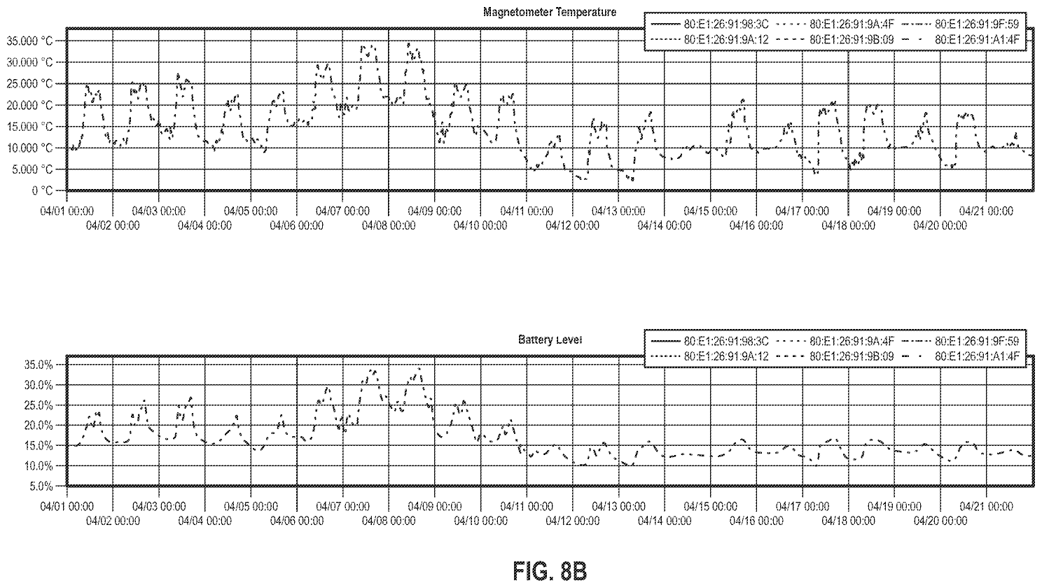

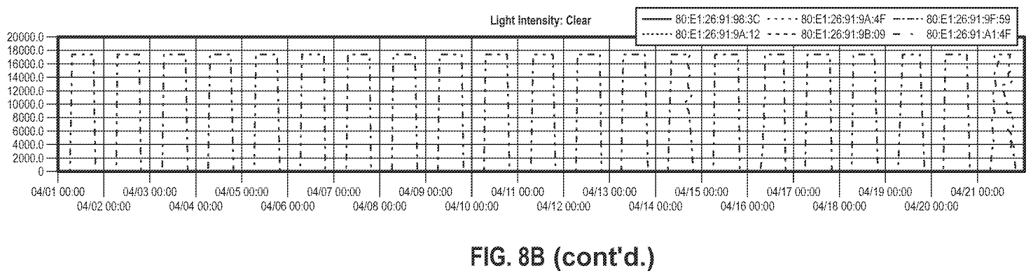

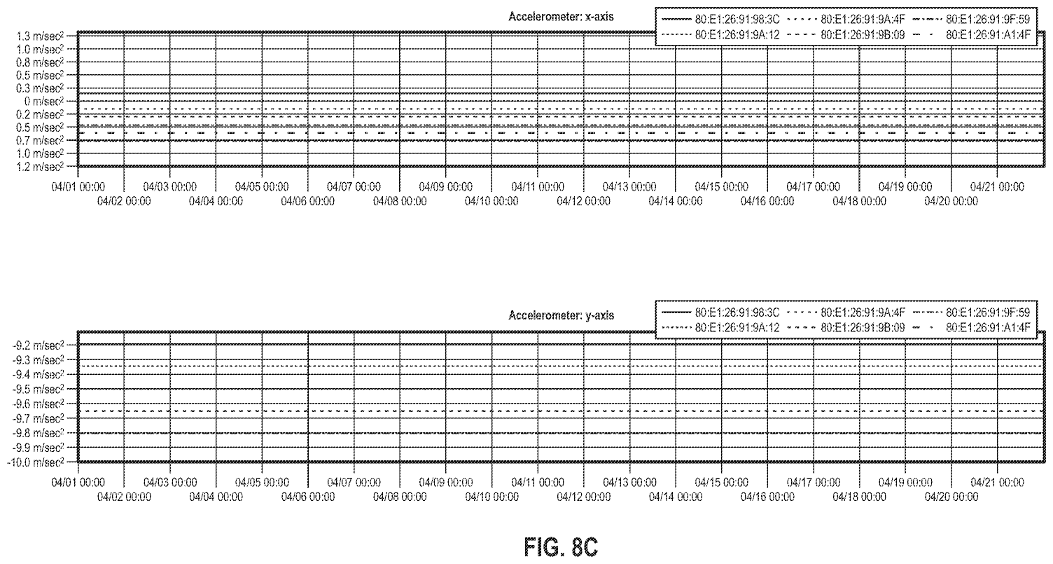

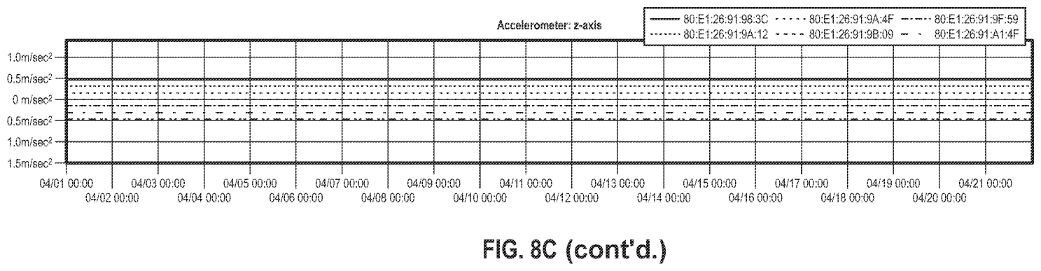

Six trees were monitored. FIG. 7A-7C shows data from a device of the present disclosure applied to each tree. FIG. 8A-8C show data from a device applied to one tree. Diameter measurements were in mm. Air temperature and magnetometer temperature measurements were in ° C. Relative humidity measurements were in % H. Battery level measurements were in %. Accelerometer measurements were in m/sec².

### Example 4: Measurement of Tree Growth

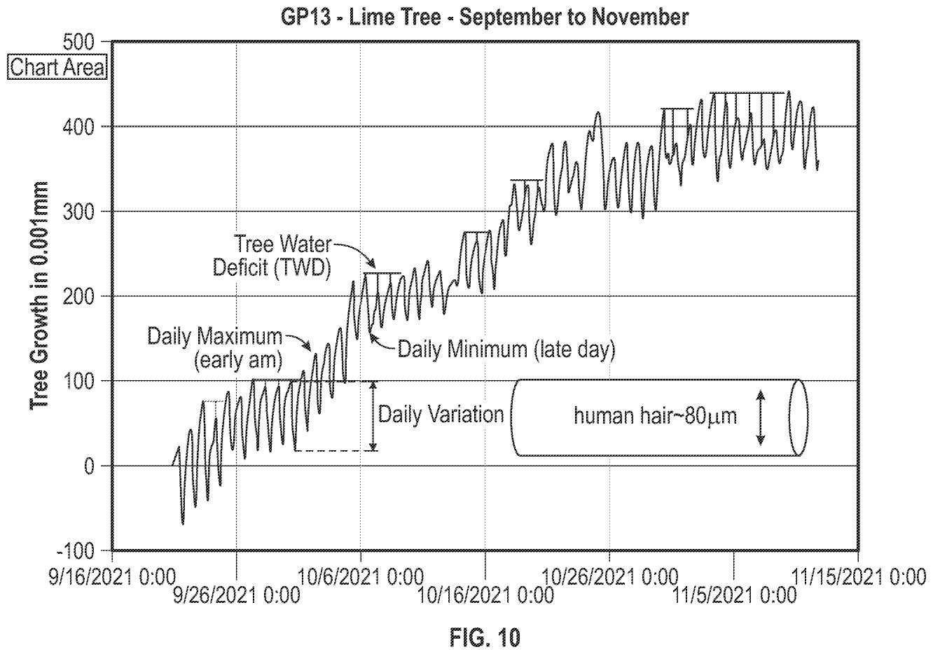

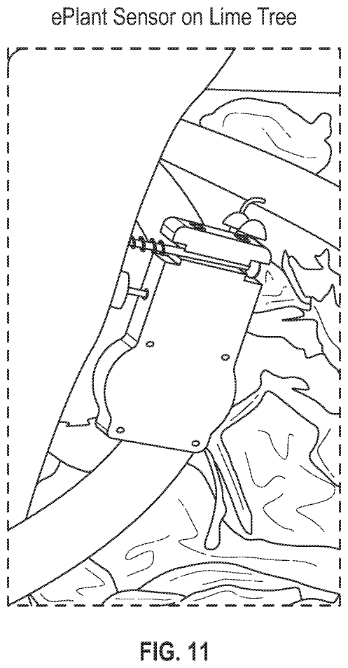

A lime tree was monitored from September 2021 to November 2021. Its growth was measured in 0.001 mm. FIG. 10 shows the daily maximum (early am), daily minimum (late day), daily variation, and tree water deficit (TWD) were monitored. The daily variation was approximately the size of a human hair (˜80 um). FIG. 11 shows the device on the lime tree. Without wishing to be bound to theory, it is thought that major drivers of daily size oscillation are related to tension generated by transpiration and the limits placed on hydraulic conductivity by the soil, sap pathways within the plant, stomatal aperture, and their respective interfaces. On the other hand, irreversible tissue expansion can be due to cell division and growth, e.g., in the meristems.

### Example 5: Water Status Monitoring in Vines and Small-Diameter Stems

Many crops may have stems or vines that are too small in diameter to accommodate a dendrometer that is affixed with a screw. However, just as with trees, it may be beneficial to monitor the size of a plant's stems and/or vines in order to optimize the plant's growing conditions. Grape vines, for example, must be grown under an optimum amount of water stress in order to produce wine grapes that have the most desirable flavor profile. Over-watered grape vines may produce watery grapes that result in an undesirable flavor profile. Under-watered grape vines may also produce grapes that have an undesirable flavor profile. In addition, under-watered grape vines may produce fewer grapes than grape vines that receive the optimum amount of water. Significant under-watering may eventually result in plant mortality. Conventional methods for monitoring the water status of grape vines may involve manually removing a grape leaf, sealing the leaf in a pressure chamber with the leaf stem protruding from the chamber, and then measuring the pressure in water that beads on the torn leaf stem. These conventional methods are typically performed just prior to a harvest; as such, even if the methods reveal that a grape vine is not receiving the optimum amount of water, there may not be enough time remaining before the harvest to correct the growing conditions in order to produce the most desirable grapes. Furthermore, the conventional methods are time consuming, require manual labor, and are prone to operator error and bias. Specifically, since a measurement only indicates the water status of a specific leaf, selecting leaves that accurately represent the status of a plant can be challenging.

The dendrometers described herein may be adapted to monitor the water status of vines and other small-diameter stems. In some embodiments, an adapted dendrometer may provide a cost-effective and automatic method to continuously measure the diameter of a wine grape vine or another plant with a small-diameter stem in order to monitor the water status (e.g., over-watered, under-watered, etc.) of said plant while the plant is growing. An adapted dendrometer according to the present disclosure may also provide growth information and environmental information that may aid the analysis of the stem diameter measurement. In some embodiments, the growth and environmental information provided by an adapted dendrometer may be used to inform crop management decisions (e.g., irrigation). In some embodiments, a user may be able to install and monitor a large number (e.g., greater than or equal to 100, 500, 1000, 5000, etc.) of adapted dendrometers in a single growing area. This may allow the user to measure a large number of plants at various locations in the growing area, which may allow the user to accurately and precisely assess growing conditions at said locations. In some embodiments, the adapted dendrometer may be used to monitor smaller, younger shoots; these shoots may provide more reliable data as they may contain less cork.

FIG. 12A depicts an exemplary dendrometer that has been adapted for measuring the diameters of vines and other small-diameter stems. Specifically, FIG. 12A illustrates a dendrometer 1200 that is affixed to a stem 1226. As shown, dendrometer 1200 may comprise a plunger 1202, a plurality of arms 1204, a housing 1206, and a pull tab 1208. Pull tab 1208 may be mechanically coupled to plunger 1202. Plunger 1202 may be retracted away from the plurality of arms 1204 by pulling pull tab 1208 away from housing 1206. In some embodiments, a user may install dendrometer 1200 on stem 1226 by retracting plunger 1202 using pull tab 1208, placing the plurality of arms 1204 on an appropriate section of stem 1226, and releasing plunger 1202 by releasing pull tab 1208. When plunger 1202 is released, it may move toward the plurality of arms 1204 and secure stem 1226 between an end of plunger 1202 and the plurality of arms 1204. In some embodiments, the plurality of arms 1204 may comprise at least 2, at least 3, at least 4, at least 5, or at least 6 arms. In some embodiments, the plurality of arms 1204 may comprise a pair of arms that extends from housing 1206 in a “V” or a “U” shape. In some embodiments, the arrangement of and shape formed by the plurality of arms 1204 may be configured to cradle stem 1226 in a kinematic determinant manner.

In some embodiments, dendrometer 1200 may be small enough to fit between closely-spaced nodes on a stem or a vine (e.g., closely-spaced nodes on a grape vine). In some embodiments, a maximum spacing between each arm of the plurality of arms 1204 may be less than or equal to 0.5, 1, 1.5, 2, 2.5, or 3 inches. In some embodiments, a maximum spacing between each arm of the plurality of arms 1204 may be greater than or equal to 0.15, 0.5, 1, 1.5, 2, or 2.5 inches. In some embodiments, the compact shape of dendrometer 1200 may minimize a measurement load path, which may increase the precision of the diameter measurements, particularly when the temperature of the environment is changing.

In some embodiments, dendrometer 1200 may be configured to attach to stems or vines having diameters less than or equal to 0.5, 1, 1.5, 2, 2.5, 3, 3.5, 4, 4.5, or 5 inches. In some embodiments, dendrometer 1200 may be configured to attach to stems or vines having diameters greater than or equal to 0.5, 1, 1.5, 2, 2.5, 3, 3.5, 4, 4.5, or 5 inches. In some embodiments, dendrometer 1200 may be configured to attach to stems or vines having diameters greater than or equal to 0.15 inches and less than or equal to 1 inch.

Dendrometer 1200 may be formed from lightweight materials. In some embodiments, housing 1206 may comprise a stable polymer such as a 30% glass-filled, UV-activated polymer that can be 3D printed by stereolithography (e.g., FormLabs Rigid10K materials). In some embodiments, housing 1206 may comprise a glass-filled polymer that can be injection molded (e.g., Noryl). In some embodiments, housing 1206 may comprise materials that are configured to transmit radio frequency signals.

In some embodiments, housing 1206 may house one or more electronic components that are configured to monitor changes in the diameter of the stem to which dendrometer 1200 is attached. Housing 1206 may comprise a removable panel 1220 which may allow a user to access the electronic components housed in housing 1206.

Additional external perspective views of dendrometer 1200 are depicted in FIG. 12E-FIG. 12M.

FIG. 12B depicts a perspective internal view of dendrometer 1200. As shown, housing 1206 may house a printed circuit assembly 1214 comprising an antenna 1216 and a magnetometer 1218. Plunger 1202 may house a magnet 1210 that is positioned at one end of a spring 1212. When a user retracts plunger 1202 by pulling pull tab 1208 away from its resting position, spring 1212 may compress. When pull tab 1208 is released, spring 1212 may be forced to re-expand, which may cause plunger 1202 to move toward the plurality of arms 1204. If the plurality of arms 1204 have been placed on a stem such as stem 1226, the movement of plunger 1202 toward the plurality of arms 1204 may be stopped by the stem.

In some embodiments, magnet 1210 may generate a magnetic field characterized by curved lines of magnetic flux. Magnetometer 1218 may be configured to measure the intensity of the magnetic field generated by magnet 1210 along at least two axes, e.g., along multiple axes, a radial axis, or a single plane. An angle of the magnetic field may be determined based on the intensity of the magnetic field along the at least two axes (e.g., along multiple axes, a radial axis, or a single plane) that is detected by magnetometer 1218. In some embodiments, the angle may be equal to or related to the arctangent of the magnetic field intensity along a first axis divided by the magnetic field intensity along a second axis. If dendrometer 1200 is affixed to a stem or a vine such as stem 1226 and the diameter of said stem/vine expands or contracts, the angle of the magnetic field generated by magnet 1210 may change. The change in the angle of the magnetic field may be related to the linear change in the diameter of the stem or vine. In some embodiments, linear change in the diameter of the stem or vine may be approximately linearly related to the change in angle of the magnetic field. In some embodiments, the linear change in the diameter may be related to the change in angle of the magnetic field by a seventh-order polynomial. In some embodiments, the linear change in diameter may be related to the change in angle of the magnetic field by a seventh-order polynomial during calibration of dendrometer 1200.

In some embodiments, spring 1212 may be configured to be strong enough to allow plunger 1202 to grip a stem or vine but weak enough to ensure that plunger 1202 does not damage the stem or vine. This may allow dendrometer 1200 to be easily attached to and removed from different stems or vines and/or different locations along a stem or vine without causing damage to the plant(s). In some embodiments, plunger 1202 may be configured to move linearly with low friction in order to allow plunger 1202 to be sensitive to small changes in the diameter of the stem or vine. In some embodiments, plunger 1202 may be sensitive to stem diameter changes on a micron scale.

In some embodiments, antenna 1216 may be configured to transmit data associated with the change in the diameter of a stem or vine to an external device (e.g., a user's computer). In some embodiments, antenna 1216 may be a radio frequency antenna. In some embodiments, antenna 1216 may be configured to wirelessly transmit data using a low-power digital radio protocol (e.g., Bluetooth LowEnergy 5 (BLE5) or LoraWAN). In some embodiments, antenna 1216 may continuously transmit data to the external device for an extended period of time (e.g., for an entire growing season).

As mentioned above, housing 1206 may comprise a removable backing 1220 that may allow a user to access printed circuit assembly 1214. Removable backing 1220 may be secured to housing 1206 using one or more fasteners 1222. In some embodiments, fasteners 1222 may comprise one or more screws, one or more bolts, and/or one or more rivets.

In some embodiments, printed circuit assembly 1214 may comprise one or more sensors in addition to magnetometer 1218. The one or more additional sensors may include a humidity sensor, a light sensor, a temperature sensor, and/or an accelerometer. A humidity sensor and an air temperature sensor may be used to determine whether changes in the diameter of the stem or vine are due to swelling in a cork layer of the stem between the plunger and the phloem. It may be necessary to distinguish between diameter changes that are due to swelling in the cork layer and expansions in the phloem since expansions in the phloem may be the actual changes of interest. In some embodiments, a humidity sensor and a temperature sensor may be used to collect information related to a potential for transpiration during photosynthesis. For example, data collected by the humidity sensor and the temperature sensor may be used to compute a Vapor Pressure Deficit. An accelerometer may help determine if dendrometer 1200 is jostled or dislocated and can provide information about the stability of the plant to which dendrometer 1200 is attached under varying wind conditions. A light sensor may be used to determine if dendrometer 1200 is in direct sunlight, to determine time of sunset and sunrise, to confirm the location of dendrometer 1200, and to provide information regarding amount of cloud cover.

In some embodiments, as shown in FIG. 12C, printed circuit assembly 1214 may receive power from a battery 1228. In some embodiments, battery 1228 may be a coin-cell battery configured to last an entire growing season. This may allow dendrometer 1200 to be installed on a stem or vine after spring pruning and removed after harvesting.

Additional internal perspective views of dendrometer 1200 are depicted in FIG. 12D and FIG. 12H.

FIGS. 12N-12P show photographs of dendrometer(s) 1200 affixed to grape vines. As shown, dendrometer 1200 may be secured to the vines using one or more elastic bands 1230. In some embodiments, elastic bands 1230 may be resistant to ultraviolet radiation. In some embodiments, a single elastic band 1230 may be stretched over a first arm of the plurality of arms 1204, around the stem, around the back side of dendrometer 1200, and over a second arm of the plurality of arms 1204.

### Example 6: Integrated Tree Sensor

A tree sensor may be configured to facilitate remote monitoring of plant health and/or growth status for multiple years without requiring maintenance after installation. The tree sensor may comprise many integrated sensors that are capable of monitoring growth status, water status, lean, and/or sway. In some embodiments, an integrated tree sensor may be configured to detect and/or account for any impacts that the sensor may have on the measurements it is making. In some embodiments, the duration that an integrated tree sensor may be installed may be limited only by the tree growth itself. An integrated tree sensor may be powered by one or more batteries that are configured to provide power for the life of the tree sensor without requiring replacement.

FIGS. 13A-13B show perspective views of an integrated tree sensor, according to some embodiments. Specifically, FIGS. 13A-13B show perspective views of an integrated tree sensor 1300 that is affixed to a trunk of a tree. Integrated tree sensor 1300 comprises a plunger 1302 and a mount screw 1304. One end of plunger 1302 may comprise a gimbal tip 1308. Over-molding 1306 may cover one or more electronic and/or control components of sensor 1300. In some embodiments, a face of sensor 1300 that faces away from the tree trunk when sensor 1300 is installed may comprise one or more solar panels 1312 that are configured to receive solar energy and convert it to electrical energy to power sensor 1300.

FIG. 13C shows a cross-sectional view of integrated tree sensor 1300. As shown, over-molding 1306 covers a single printed circuit board 1324. In some embodiments, printed circuit board 1324 may be configured to support all mechanical and electrical components of sensor 1300 (i.e., all components of sensor 1300 may be affixed to printed circuit board 1324). Electronic components of sensor 1300 may include one or more antennas such as a LORA antenna 1326 and a NFC antenna 1332. In some embodiments, these antennas may be configured to transmit data over long ranges while consuming small amounts of power.

In some embodiments, printed circuit board 1324 may comprise materials having stable structural properties and low coefficients of thermal expansion compared to injection molded plastics. In some embodiments, printed circuit board 1324 may comprise laminated layers of an epoxy-fiberglass composite (e.g., G10 or FR4).

In some embodiments, over-molding 1306 may be configured to hermetically seal printed circuit board 1324. Over-molding 1306 may be applied using a low-pressure over-molding system (e.g., Techno-Melt by Henkel). Over-molding 1306 may be configured to protect one or more electronic components of integrated tree sensor from exposure to water and other contaminants. In some embodiments, over-molding 1306 may be applied in such a way that one or more components of sensor 1300 remain exposed.

In some embodiments, mount screw 1304 may be configured to securely affix sensor 1300 to a tree trunk. Mount screw 1304 may be a button-head screw and may comprise stainless steel, brass, aluminum, and/or titanium. Mount screw 1304 may be the only screw that is needed to affix sensor 1300. Using a single screw may facilitate easy and efficient installation of sensor 1300, since a single screw only requires a single hole to be drilled in the tree trunk. In order to ensure sensor 1300 makes stable measurements over extended periods of time, it may be necessary for the screw joint of mount screw 1304 to be tight and secure.

In some embodiments, a compression limiter 1322 may be installed in printed circuit board 1324 in order to provide a durable interface between screw 1306 and printed circuit board 1324. Compression limiter 1322 may be a metal collar and may be installed in printed circuit board 1324 using automatic soldering equipment. After a hole for mount screw 1304 has been drilled into a tree trunk, sensor 1300 may be affixed to the trunk by passing mount screw 1304 into a front face of sensor 1300, through compression limiter 1322 and printed circuit board 1324, and out of a back face of sensor 1300. A nut 1316 may be installed on a tail end of mount screw 1304. Mount screw 1324 may be inserted an appropriate depth into the hole in the tree trunk. Plunger 1302 may then be aligned. Once plunger 1302 has been aligned, nut 1316 may be tightened from the side using a wrench (e.g., a crescent wrench) in order to prevent axial movement of mount screw 1324.

In some embodiments, a mount hole or slot in printed circuit board 1324 may be exposed in order to allow screw 1304 to affix sensor 1300 to the tree. In some embodiments, mount screw 1304 may be a threaded rod comprising a nut that has been pre-fixed to the rod using an adhesive, solder, or welding. In some embodiments, the nut may be machined as part of the threaded rod. After sensor 1300 has been appropriately placed, a second nut may be installed and tightened from the front face of sensor 1300. This may allow sensor 1300 to be installed and removed without fully removing mount screw 1304 from the tree.

FIG. 13D shows a cross-sectional view of plunger 1302. Plunger 1302 may house a magnet 1328. In some embodiments, magnet 1328 may comprise neodymium. Magnet 1328 may generate a magnetic field. When sensor 1300 is installed on a tree trunk, changes in the diameter of the tree trunk may impact physical properties of the magnetic field generated by magnet 1328. Sensor 1300 may comprise a magnetometer 1334 that is configured to detect changes in the magnetic field generated by magnet 1328. In some embodiments, the magnetic field generated by magnet 1328 may be characterized by a curved magnetic field path that changes angle relative to a fixed point as plunger 1302 moves in and out as a result of changes in the diameter of the tree trunk. Magnetometer 1334 may measure the intensity of the magnetic field in two orthogonal axes. Based on the measured intensities, the angle of the magnetic field lines relative to a fixed point can be calculated. This angle can be related to the linear position of plunger 1302. In some embodiments, the linear position of plunger 1302 may be determined to micron resolution. In some embodiments, the characteristics of the magnetic field that is produced by magnet 1328 may be resistant to change over the life of sensor 1300, provided sensor 1300 is not heated artificially.

In some embodiments, plunger 1302 may be partially housed in a guide 1318. A spring 1330 may surround plunger 1302 within guide cap 1318. In some embodiments, plunger 1302 may be installed by pulling back a plunger cap 1310 in order to compress spring 1330 and then releasing plunger cap 1310 in order to cause plunger 1302 to make contact with the trunk of a tree. In some embodiments, an anti-rotation pin 1320 may be positioned at one end of spring 1330 within guide cap 1318 in order to prevent plunger 1302 from rotating and to facilitate the transfer of the spring force to plunger 1302.

As mentioned above, plunger 1302 may comprise a gimbal tip 1308. Gimbal tip 1308 may be configured to permit plunger 1302 to pivot about an axis. In some embodiments, gimbal tip 1308 may be configured to provide a contact area of reasonable size between plunger 1302 and the tree trunk to which sensor 1300 is affixed. In some embodiments, the surface area of gimbal tip 1308 may be greater than or equal to 5, 10, 15, 20, 25, 30, 35, 40, 45, or 50 square millimeters. In some embodiments, the surface area of gimbal tip 1308 may be less than or equal to 1000, 500, 200, 100, 90, 80, or 70 square millimeters. In some embodiments, the surface area of gimbal tip 1308 may be between 10-50, 10-100, 10-500, 10-1000, or 10-1500 square millimeters. In some embodiments, one end of plunger 1302 may comprise a spherical ball point. Gimbal tip 1308 may comprise a spherical cavity configured to receive the spherical ball point of plunger 1302. In some embodiments, gimbal tip 1308 may be less than or equal to 5, 4, 3, 2, or 1 mm thick. In some embodiments, gimbal tip 1308 may be greater than or equal to 0.5, 1, 2, 3, or 4 mm thick. In some embodiments, gimbal tip 1308 may be formed via injection molding and may comprise plastic (e.g., low-friction plastic such as acetal or PETG). FIG. 13P shows perspective views of gimbal tip 1308.

In some embodiments, solar panel 1312 may be a component of a hybrid capacitor/lithium battery 1336 and charge control circuit that is integrated on printed circuit board 1324 and is configured to maximize energy collection for sensor 1300. Solar panel 1312 may be configured to provide power to sensor 1300 for the life of sensor 1300. In some embodiments, sensor 1300 may be configured to operate for an extended period of time (e.g., days or weeks) in darkness using power that was collected by solar panel 1312 and stored on hybrid capacitor 1336.

FIG. 13Q shows an internal, cross-sectional view of integrated tree sensor 1300 mounted to an alumina-silicate ceramic plate used to characterize temperature and humidity sensitivity (in operation, sensor 1300 would be mounted to a plant part as described herein). In this illustration, printed circuit board 1324 is a PCA-00012A screwed to the housing, which in this example is made from Rigid 10K glass-filled resin. Magnetometer 1334 is attached to the PCA 1324, which can also include a variety of other sensors, e.g., as described herein. The sensor includes magnet 1328, which can be a neodymium cylinder magnet such as D34-N52 (K&J Magnetics, Inc.). Mount screw 1304 is mounted to the ceramic plate with nuts 1316 and 1318 on either side of the plate, respectively. Plunger 1302 (18-8 SS shaft) rests on the ceramic plate with tip 1308, which can be made from a plastic such as DELRIN® polyoxymethylene (POM) polymer resin. A shuttle (in this example, made from Rigid 4000 resin) is press-fitted onto the shaft of plunger 1302, and mount screw 1304 is held in place via clamp (in this example, made from Rigid 10K glass-filled resin).

In some embodiments, sensor 1300 may comprise additional sensors that are configured to collect additional data related to the health and growth of a tree trunk. In some embodiments, sensor 1300 may comprise a three-axis accelerometer configured to measure changes in the tilt of the tree trunk over long (i.e., days or longer) periods of time (“lean”). In some embodiments, the accelerometer may be configured to detect movement of the tree trunk over short periods of time (“sway”). In some embodiments, the accelerometer may be configured to detect sharp accelerations of the tree trunk (“impact”). In some embodiments, sensor 1300 may comprise a temperature sensor. Temperature sensor may monitor changes in temperature that may introduce errors into the measurement of trunk diameter.

Alternative mounting hardware is illustrated in FIGS. 14A-14C. For simplicity, only mounting elements are shown in FIGS. 14A-14C. Advantageously, the sensors of the present disclosure can utilize a variety of mounting options to attach to a variety of different tree types and situations. The mounting is secure for precise measurements over long periods of time because of, inter alia, the high contact forces and metal-to-metal interface between the nut or screw faces and compression limiter. In some embodiments, a high-strength solder joint between the compression limiter and the G10/FR4 PCB that in turn secures the magnetometer and accelerometer results in a simple, stable measurement platform. There are no plastic parts or friction grips in this critical measurement load path. Achieving an easy, secure attachment to the tree using just one screw hole is an advantage relative to other approaches that may require multiple holes to be drilled in a tree and the need there would be to achieve precise alignment between those multiple holes.

FIG. 14A shows integrated tree sensor 1400 and its printed circuit board 1402 with mounting hardware including a captive screw and re-adjustable mount screw. Captive screw 1408 is retained in the device assembly by retainer ring 1406 that has an interference fit with the ID of compression limiter 1404 and a loose fit around a narrow portion of the captive screw 1408. This may be a plastic ring with a slit to allow it to be installed on the captive screw, or it could be a washer, o-ring or other similar shape or the compression limiter could have a feature that tends to keep the screw from falling out. The captive screw may be convenient for installers, eliminating the possibility of dropping nuts or other small items in the leaves and dirt around the tree base. In some embodiments, captive screw 1408 has a button head with a hex socket to engage a tightening wrench. In some embodiments, captive screw 1408 has a knurled or flanged shape to allow tightening without a tool. In some embodiments, captive screw 1408 has a tamper resistant drive, e.g., to make it more difficult for unauthorized people to remove.

Device 1400 is mounted onto the tree trunk by mount screw 1410. A hole is typically drilled in the tree at the mounting area and, particularly if thick bark is present, some of the cork may be removed in the mounting area. In some embodiments, mount screw 1410 is self-threading so that no hole needs to be drilled, or mount screw 1410 comprises a nail-like shape with raised features to improve grip and is configured to be pressed in by a nail-gun, hammer or other insertion tool.

In some embodiments, mount screw 1410 has machined threads (M5×0.8 shown) for a portion and a smooth portion closer to the head. The length of the smooth portion is such that it indicates the correct installation depth, and it is narrow enough that the growing screw will not tend to push the screw out and will fill in the space around the screw that the threads may engage when the screw is backed out later. Alternatively, mount screw 1410 may be threaded all the way or closer to the head. In some embodiments, the head of mount screw 1410 has a hexagonal nut flange, where the distal face provides a flat surface that the proximal face of compression limiter 1404 rests on. This nut shape enables mount screw 1410 to be inserted into the tree using a standard nut driver. In some embodiments, the distal end of mount screw 1410 has a cylindrical shaped protrusion to locate compression limiter 1404 and female threads to receive captive screw 1408.

In some embodiments, a data monitoring system of integrated tree sensor 1400 may alert operators when the tree has grown to the point where the plunger is near the end of the stroke, and at this point integrated tree sensor 1400 can be easily adjusted to continue at the start of the plunger stroke again. Captive screw 1408 is loosened, then mount screw 1410 is unscrewed until the threaded portion is just visible, and integrated tree sensor 1400 is reinstalled by tightening captive screw 1408.

FIG. 14B shows integrated tree sensor 1400 and its printed circuit board 1402

with mounting hardware including threaded rod 1420 and nuts 1422 and 1424. In some embodiments, threaded rod 1420 (which in some embodiments could be a set screw) may have nut 1422 pre-installed at the correct location and may be bonded in place, e.g., using a bonding adhesive (such as LOCTITE® bonding adhesive), brazing, soldering, or welding. In some embodiments, threaded rod 1420 and nut 1422 are made as a solid piece of hardware. Integrated tree sensor 1400 may then be placed onto threaded rod 1420 and secured by nut 1424 on the distal side. In some embodiments, outer nut 1424 may be a thumb-nut that is knurled or tabbed so that it can be inserted without tools.

FIG. 14C shows integrated tree sensor 1400 and its printed circuit board 1402 with mounting hardware including a long threaded-rod. On trees where significant growth is expected to occur, it may be desirable to mount integrated tree sensor 1400 using long threaded-rod (e.g., 1432 in FIG. 14C) that allows the device to be relocated easily without turning the screw relative to the tree. As shown in the top panel of this exemplary scenario, upon initial installation, plunger 1430 of integrated tree sensor 1400 is approximately 1 mm from full extension. After passage of time and growth of the tree trunk (FIG. 14C, middle panel), plunger 1430 is now nearly fully depressed after about 12 mm of radial growth of the tree trunk. Nuts 1434 and 1436 are used to secure integrated tree sensor 1400 to threaded-rod 1432, and both may be adjusted to move integrated tree sensor 1400 away from the tree after it has grown. As shown in the bottom panel of FIG. 14C, nuts 1434 and 1436 are adjusted while leaving threaded rod 1432 as it was. After adjustment, plunger 1430 is approximately 1 mm from full extension again, as it was in initial installation (FIG. 14C, top panel).

Various amounts of plunger travel are possible with some trade-offs. With the geometry shown a single ¼″ long magnet will produce a magnetic field at the magnetometer that has similar magnitude while rotating about 300 degrees as the plunger moves linearly over about 12 mm of travel. Smaller geometry would produce the same rotation over a smaller amount of travel and could result in an even higher measurement sensitivity. Larger geometry would result in lower sensitivity and greater travel. To achieve both long travel and high sensitivity it is possible to use a magnet arrangement of several alternating north and south poles that produces continuous rotation of the magnetic field beyond 360 degrees repeating for as many pole pairs as one provides in the plunger. A longer support structure and spring arrangement would also be needed. In some embodiments, the single magnet and 12 mm working measurement range is a practical compromise that results in sufficient measurement sensitivity and workable re-adjustment time periods for many tree types and applications.

### Example 7: Tracking Change in Tree Lean Using an Integrated Tree Sensor

As disclosed herein, an integrated sensor of the present disclosure can include an accelerometer, e.g., for measuring, tracking, or detecting tree lean or falling off trees or parts thereof (such as limbs).

Two integrated tree sensors were mounted next to each other on a leaning part of a citriodora eucalyptus tree. FIGS. 15A & 15B show exemplary accelerometer data obtained from the sensors. FIG. 15A shows lean over time, including deviation from the x-axis and y-axis over time. FIG. 15B shows pitch and roll angle (in degrees) over time from the two sensors. These data have been corrected for temperature. The fact that both sensors were in such close agreement suggests that the measurements are accurate, and indicates progression of tree lean where the roll angle has gone from the tare value of 0 to approximately −0.3 degrees during the observation period. In some embodiments, if the tree progresses beyond a certain degree of change (e.g., beyond 1.0 degree), an alert could be triggered by the integrated sensor that the tree, or a part of the tree (e.g., a branch), may be at risk of falling.

What is claimed is:

1. A sensor for measuring plant part size and/or other plant part characteristics, comprising:
a) one or more fasteners configured to be positioned in or around a plant part;
b) two or more components selected from the group consisting of: a dendrometer, an accelerometer, an air temperature sensor, a humidity sensor, and a light sensor;
c) a processor; and
d) a power supply.

2. The sensor of claim 1, wherein the processor comprises a printed circuit board (PCB).

3. The sensor of claim 2, wherein one or both of the two or more components is/are affixed to the PCB.

4. The sensor of claim 3, wherein all of the two or more components are affixed to the PCB.

5. The sensor of any one of claims 2-4, wherein the PCB comprises an epoxy-fiberglass composite material.

6. The sensor of any one of claims 1-5, wherein the power supply comprises a battery.

7. The sensor of any one of claims 1-6, wherein the power supply comprises a solar panel.

8. The sensor of claim 6 or claim 7, wherein the power supply comprises an integrated solar panel, hybrid capacitor, and lithium battery.

9. The sensor of claim 6, wherein the battery is a coin cell battery.

10. The sensor of any one of claims 6-9, wherein the processor comprises a PCB, and wherein the battery is affixed to the PCB.

11. The sensor of any one of claims 7-9, wherein the processor comprises a PCB, and wherein the solar panel is affixed to the PCB.

12. The sensor of any one of claims 1-11, further comprising a housing that encloses at least the processor and power supply.

13. The sensor of claim 12, wherein the housing is or comprises molded plastic.

14. The sensor of claim 12 or claim 13, wherein the housing is a single piece of overmolded plastic that lacks a seal, junction, or fastener.

15. The sensor of claim 14, wherein the housing further comprises an O-ring.

16. The sensor of any one of claims 12-15, wherein the housing is or comprises a polymer resin.

17. The sensor of any one of claims 13-16, wherein the plastic or polymer resin is glass-filled.

18. The sensor of claim 17, wherein the plastic or polymer resin is 10-40% glass.

19. The sensor of claim 18, wherein the plastic or polymer resin is 30% glass.

20. The sensor of any one of claims 1-19, wherein the sensor comprises a dendrometer.

21. The sensor of claim 20, wherein the dendrometer comprises:
1) a plunger having a cap and a shaft, wherein the cap is configured to be positioned against the plant part, and wherein the plunger is configured to move laterally in proportion to a change in plant size when the cap is positioned against the plant part;
2) a magnet attached to or within the shaft, wherein the magnet is configured to move laterally in association with the plunger; and
3) a magnetometer configured to detect position of the magnet.

22. The sensor of claim 21, wherein the magnetometer is configured to detect position of the magnet along multiple axes, a radial axis, or a single plane.

23. The sensor of claim 21 or claim 22, wherein the magnetometer is configured to detect position of the magnet at micron-scale resolution.

24. The sensor of any one of claims 21-23, wherein the magnet is neodymium magnet.

25. The sensor of any one of claims 21-24, wherein the processor comprises a PCB, and wherein the magnetometer is affixed to the PCB.

26. The sensor of any one of claims 1-25, wherein the sensor is configured to measure change in diameter or radius of the plant part.

27. The sensor of any one of claims 1-26, wherein the sensor is configured to measure plant part size multiple times per day.

28. The sensor of claim 27, wherein the sensor is configured to measure plant part size at an interval of 15 minutes or less.

29. The sensor of claim 27, wherein the sensor is configured to measure plant part size at an interval of 5 minutes or less.

30. The sensor of claim 27, wherein the sensor is configured to measure plant part size at an interval of 5 seconds.

31. The sensor of any one of claims 1-30, wherein the sensor comprises an accelerometer.

32. The sensor of claim 31, wherein the accelerometer is a 3-axis accelerometer.

33. The sensor of claim 31 or claim 32, wherein the processor comprises a PCB, and wherein the accelerometer is affixed to the PCB.

34. The sensor of any one of claims 1-33, wherein the sensor comprises an air temperature sensor.

35. The sensor of claim 34, wherein the processor comprises a PCB, and wherein the air temperature sensor is affixed to the PCB.

36. The sensor of any one of claims 1-35, wherein the sensor comprises a humidity sensor.

37. The sensor of claim 36, wherein the processor comprises a PCB, and wherein the humidity sensor is affixed to the PCB.

38. The sensor of any one of claims 1-37, wherein the sensor comprises a light sensor.

39. The sensor of claim 38, wherein the processor comprises a PCB, and wherein the light sensor is affixed to the PCB.

40. The sensor of any one of claims 1-39, wherein the sensor comprises a dendrometer and one or more of: an accelerometer, an air temperature sensor, a humidity sensor, and a light sensor.

41. The sensor of claim 40, wherein the sensor comprises a dendrometer, an accelerometer, an air temperature sensor, a humidity sensor, and a light sensor.

42. The sensor of any one of claims 1-41, further comprising a transmitter.

43. The sensor of claim 42, wherein the transmitter is a Bluetooth radio or transceiver.

44. The sensor of claim 43, wherein the Bluetooth radio or transceiver is a Bluetooth Low Energy (BLE) radio or transceiver.

45. The sensor of claim 42, wherein the transmitter is a Long Range (LoRa) transceiver.

46. The sensor of claim 42, wherein the transmitter is a Near Field Communication (NFC) transceiver.

47. The sensor of any one of claims 42-46, wherein the processor comprises a PCB, and wherein the transmitter is affixed to the PCB

48. The sensor of any one of claims 1-47, wherein the one or more fasteners comprises a screw, threaded rod, or nail, and wherein the screw, threaded rod, or nail is configured to be positioned within the plant part and mount the sensor to the plant part.

49. The sensor of any one of claims 1-47, wherein the one or more fasteners comprises one or more curved arm(s), wherein the curved arm(s) are configured to be positioned around the plant part.

50. The sensor of claim 49, wherein the one or more fasteners comprises two curved arms arranged in a V-shape.

51. The sensor of claim 49 or claim 50, wherein the curved arm(s) are configured to be positioned around the plant part.

52. The sensor of any one of claims 47-51, wherein the one or more fasteners further comprises an elastic band configured to be wrapped around the sensor and the plant part.

53. The sensor of any one of claims 47-52, wherein the one or more fasteners comprises a screw, wherein the processor comprises a PCB, and wherein the screw is affixed to the PCB.

54. The sensor of claim 53, wherein the PCB comprises a compression-limiting element around the screw.

55. The sensor of any one of claims 21-54, wherein the plunger cap further comprises a gimbal.

56. The sensor of any one of claims 21-55, wherein the plunger cap is or comprises molded plastic.

57. The sensor of any one of claims 21-56, wherein the plunger cap is less than about 3 mm in thickness.

58. The sensor of any one of claims 21-57, wherein the plunger cap is configured to contact the plant part over a surface area of between about 10 mm² and about 100 mm².

59. The sensor of any one of claims 21-58, further comprising a spring around or affixed to the plunger.

60. The sensor of any one of claims 21-59, further comprising a pull tab attached to the plunger shaft opposite the plunger cap.

61. The sensor of any one of claims 21-60, wherein the plunger shaft comprises aluminum or stainless steel.

62. The sensor of claim 61, wherein the plunger shaft is a hollow cylinder, and the magnet is a cylindrical magnet positioned inside the plunger shaft.

63. The sensor of any one of claims 48-62, wherein the screw, threaded rod, or nail comprises stainless steel, brass, aluminum, or titanium.

64. The sensor of any one of claims 48-63, wherein the one or more fasteners comprises a screw, and wherein the sensor further comprises a nut configured to be positioned around the screw between the sensor and the plant part.

65. The sensor of claim 64, further comprising a second nut configured to be positioned around the screw on a face of the sensor distal to the plant part.

66. The sensor of any one of claims 48-65, wherein the one or more fasteners comprises a screw having a first end and a second end, wherein the sensor further comprises:
(i) a compression-limiting element having a first opening and a second opening; and
(ii) a captive screw;
wherein the first end of the screw is configured to be positioned within the plant part and mount the sensor to the plant part;
wherein the first opening of the compression-limiting element is configured to receive the second end of the screw; and
wherein the second opening of the compression-limiting element is configured to receive the captive screw.

67. The sensor of claim 66, further comprising a retaining ring configured to be positioned around the captive screw.

68. The sensor of any one of claims 48-63, wherein the one or more fasteners comprises a threaded rod, and wherein the sensor further comprises a first nut configured to be positioned around the threaded rod between the plant part and the sensor and a second nut configured to be positioned around the threaded rod adjacent to the sensor and distal to the plant part.

69. The sensor of any one of claims 21-68, further comprising a hollow shuttle positioned around the plunger shaft.

70. The sensor of any one of claims 1-69, wherein the plant is a tree or woody plant.

71. The sensor of claim 70, wherein the plant part is a stem, trunk, bole, or branch.

72. The sensor of claim 70 or claim 71, wherein the plant is a crop tree.

73. The sensor of any one of claims 70-72, wherein the plant is a citrus, olive, nut, cacao, oak, pine, redwood, or maple tree.

74. The sensor of any one of claims 1-69, wherein the plant is a vine.

75. The sensor of claim 74, wherein the plant part is a trunk, shoot, branch, cane, fruit, or stem.

76. The sensor of claim 74 or claim 75, wherein the vine is a grape vine.

77. A system for measuring plant part size and/or other plant part characteristics, comprising:
a) a sensor according to any one of claims 1-76; and
b) a mobile device and/or server;
wherein the sensor is connected to the mobile device and/or server via wireless communication and configured to transmit data to the mobile device and/or server.

78. The system of claim 77, wherein the sensor is connected to the mobile device and/or server via Bluetooth low energy (BLE), Long Range (LoRa), Near Field Communication (NFC), or a combination thereof.

79. The system of claim 77 or claim 78, comprising a mobile device, wherein the sensor is configured to transmit data to the mobile device.

80. The system of any one of claims 77-79, comprising a server, wherein the sensor is configured to transmit data to the server.

81. The system of any one of claims 77-80, wherein the sensor is configured to transmit data related to one or more of: the magnetometer, plant part size, wireless communication signal strength, accelerometer, light sensor, humidity sensor, air temperature sensor, or a combination thereof to the mobile device and/or server.

82. The system of any one of claims 77-81, wherein the mobile device comprises a Global Positioning System (GPS) sensor, and wherein the GPS sensor is configured to obtain location information using the GPS sensor and associate the location information with the sensor.

83. The system of any one of claims 77-82, wherein the mobile device comprises a camera or other image sensor.

84. The system of any one of claims 77-83, comprising a plurality of sensors according to any one of claims 1-75; wherein each sensor of the plurality is connected to the mobile device and/or server via wireless communication and configured to transmit data to the mobile device and/or server.

85. A system for measuring plant part size and/or other plant part characteristics of a plurality of plants, comprising a plurality of sensors according to any one of claims 1-76; wherein each sensor of the plurality is configured to measure plant part size and/or other plant part characteristics of a single plant of the plurality.

86. The system of claim 85, further comprising a mobile device; wherein each sensor of the plurality is connected to the mobile device and configured to transmit data to the mobile device.

87. The system of claim 85 or claim 86, further comprising a server; wherein each sensor of the plurality is connected to the server and configured to transmit data to the mobile device.

88. A method for measuring size of a plant part and/or other plant part characteristics, comprising:
a) affixing a sensor according to any one of claims 1-76 to the plant part; and
b) measuring size and/or other plant part characteristics of the plant part based at least in part on data collected from the two or more components of the sensor.

89. The method of claim 88, wherein the size and/or other plant part characteristics of the plant part is measured at a first time, and wherein the method further comprises measuring size and/or other plant part characteristics of the plant part at a second time different from the first time, wherein the measurement of size and/or other plant part characteristics at the second time is based at least in part on data collected from the two or more components of the sensor.
